SEQUENCE LISTING (4 sequences)

SEQ ID NO: 1 - DNA, 2820 bp, Homo sapiens
  misc_feature (866)..(866): n is a, c, g, or t
aaaactgacccccaaccccctaacactcgaagatagggccgtatcactaccccgcccggccccgtaacccccccccgccccggcccggaattttgcaaatcggcgaccccgcgtccggttgcggtggctgaggaggcccgagaggagtcggtggcagcggcggcggcgggaccggcagcagcagcagcagcagcagcagcaaccactagcctcctgccccgcggcgctgccgcacgagccccacgagccgctcaccccgccgttctcagcgctgcccgaccccgctggcgcgccctcccgccgccagtcccggcagcgccctcagttgtcctccgactcgccctcggccttccgcgccagccgcagccacagccgcaacgccacccgcagccacagccacagccacagccccaggcatagccttcggcacagccccggctccggctcctgcggcagctcctctgggcaccgtccctgcgccgacatcctggaggttgggatgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggnccgagccggtgcaagatctcttcgacttcatcacggaaaggggagccctgcaagaggagctggcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatagcagcctttctggcaggtcctcccctctcttgtcagatgcccgagggaggggaagcttctgtctccagcttcccgagtaccagtgacacgtctcgccaagcaggacagtgcttgatacaggaacaacatttacaactcattccagatcccaggcccctggaggctgcctcccaacagtggggaagagtgactctccaggggtcctaggcctcaactcctcccatagatactctcttcttctcataggtgtccagcattgctggactctgaaatatcccgggggtggggggtgggggtgggtcagaaccctgccatggaactgttttcttcatcatgagttctgctgaatgccgcgatgggtcaggtaggggggaaacaggttgggatgggataggactagcaccattttaagtccctgtcacctcttccgactctttctgagtgccttctgtggggactccggctgtgctgggagaaatacttgaacttgcctcttttacctgctgcttctccaaaaatctgcctgggttttgttccctatttttctctcctgtcctccctcaccccctccttcatatgaaaggtgccatggaagaggctacagggccaaacgctgagccacctgcccttttttctgcctcctttagtaaaactccgagtgaactggtcttcctttttggtttttacttaactgtttcaaagccaagacctcacacacacaaaaaatgcacaaacaatgcaatcaacagaaaagctgtaaatgtgtgtacagttggcatggtagtatacaaaaagattgtagtggatctaatttttaagaaattttgcctttaagttattttacctgtttttgtttcttgttttgaaagatgcgcattctaacctggaggtcaatgttatgtatttatttatttatttatttggttcccttcctattccaagcttccatagctgctgccctagttttctttcctcctttcctcctctgacttggggaccttttgggggagggctgcgacgcttgctctgtttgtggggtgacgggactcaggcgggacagtgctgcagctccctggcttctgtggggcccctcacctacttacccaggtgggtcccggctctgtgggtgatggggaggggcattgctgactgtgtatataggataattatgaaaagcagttctggatggtgtgccttccagatcctctctggggctgtgttttgagcagcaggtagcctgctggttttatctgagtgaaatactgtacaggggaataaaagagatcttatttttttttttatacttggcgttttttgaataaaaaccttttgtcttaaaaaaaaaaaaaaaaaaaaaaaaaaaaaaaaaaaaaaaaaaaa

SEQ ID NO: 2 - RNA, 2820 nt, Homo sapiens
  misc_feature (866)..(866): n is a, c, g, or u
aaaacugacccccaacccccuaacacucgaagauagggccguaucacuaccccgcccggccccguaacccccccccgccccggcccggaauuuugcaaaucggcgaccccgcguccgguugcgguggcugaggaggcccgagaggagucgguggcagcggcggcggcgggaccggcagcagcagcagcagcagcagcagcaaccacuagccuccugccccgcggcgcugccgcacgagccccacgagccgcucaccccgccguucucagcgcugcccgaccccgcuggcgcgcccucccgccgccagucccggcagcgcccucaguuguccuccgacucgcccucggccuuccgcgccagccgcagccacagccgcaacgccacccgcagccacagccacagccacagccccaggcauagccuucggcacagccccggcuccggcuccugcggcagcuccucugggcaccgucccugcgccgacauccuggagguugggaugcucuuguccaaaaucaacucgcuugcccaccugcgcgccgcgcccugcaacgaccugcacgccaccaagcuggcgcccggcaaggagaaggagccccuggagucgcaguaccaggugggcccgcuacugggcagcggcggcuucggcucggucuacucaggcauccgcgucuccgacaacuugccgguggccaucaaacacguggagaaggaccggauuuccgacuggggagagcugccuaauggcacucgagugcccauggaagugguccugcugaagaaggugagcucggguuucuccggcgucauuaggcuccuggacugguucgagaggcccgacaguuucguccugauccuggagaggnccgagccggugcaagaucucuucgacuucaucacggaaaggggagcccugcaagaggagcuggcccgcagcuucuucuggcaggugcuggaggccgugcggcacugccacaacugcggggugcuccaccgcgacaucaaggacgaaaacauccuuaucgaccucaaucgcggcgagcucaagcucaucgacuucgggucgggggcgcugcucaaggacaccgucuacacggacuucgaugggacccgaguguauagcccuccagaguggauccgcuaccaucgcuaccauggcaggucggcggcagucuggucccuggggauccugcuguaugauaugguguguggagauauuccuuucgagcaugacgaagagaucaucaggggccagguuuucuucaggcagagggucucuucagaaugucagcaucucauuagauggugcuuggcccugagaccaucagauaggccaaccuucgaagaaauccagaaccauccauggaugcaagauguucuccugccccaggaaacugcugagauccaccuccacagccugucgccggggcccagcaaauagcagccuuucuggcagguccuccccucucuugucagaugcccgagggaggggaagcuucugucuccagcuucccgaguaccagugacacgucucgccaagcaggacagugcuugauacaggaacaacauuuacaacucauuccagaucccaggccccuggaggcugccucccaacaguggggaagagugacucuccagggguccuaggccucaacuccucccauagauacucucuucuucucauagguguccagcauugcuggacucugaaauaucccggggguggggggugggggugggucagaacccugccauggaacuguuuucuucaucaugaguucugcugaaugccgcgaugggucagguaggggggaaacagguugggaugggauaggacuagcaccauuuuaagucccugucaccucuuccgacucuuucugagugccuucuguggggacuccggcugugcugggagaaauacuugaacuugccucuuuuaccugcugcuucuccaaaaaucugccuggguuuuguucccuauuuuucucuccuguccucccucacccccuccuucauaugaaaggugccauggaagaggcuacagggccaaacgcugagccaccugcccuuuuuucugccuccuuuaguaaaacuccgagugaacuggucuuccuuuuugguuuuuacuuaacuguuucaaagccaagaccucacacacacaaaaaaugcacaaacaaugcaaucaacagaaaagcuguaaauguguguacaguuggcaugguaguauacaaaaagauuguaguggaucuaauuuuuaagaaauuuugccuuuaaguuauuuuaccuguuuuuguuucuuguuuugaaagaugcgcauucuaaccuggaggucaauguuauguauuuauuuauuuauuuauuugguucccuuccuauuccaagcuuccauagcugcugcccuaguuuucuuuccuccuuuccuccucugacuuggggaccuuuugggggagggcugcgacgcuugcucuguuuguggggugacgggacucaggcgggacagugcugcagcucccuggcuucuguggggccccucaccuacuuacccaggugggucccggcucugugggugauggggaggggcauugcugacuguguauauaggauaauuaugaaaagcaguucuggauggugugccuuccagauccucucuggggcuguguuuugagcagcagguagccugcugguuuuaucugagugaaauacuguacaggggaauaaaagagaucuuauuuuuuuuuuuauacuuggcguuuuuugaauaaaaaccuuuugucuuaaaaaaaaaaaaaaaaaaaaaaaaaaaaaaaaaaaaaaaaaaaa

SEQ ID NO: 3 - RNA, 19 nt, Homo sapiens
gcaggacagugcuugauac

SEQ ID NO: 4 - protein, 404 aa, Homo sapiens
  misc_feature (214)..(214): Xaa can be any naturally occurring amino acid
LPHEPHEPLTPPFSALPDPAGAPSRRQSRQRPQLSSDSPSAFRASRSHSRNATRSHSHSHSPRHSLRHSPGSGSCGSSSGHRPCADILEVGMLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFERPDSFVLILERXEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWMQDVLLPQETAEIHLHSLSPGPSK

(19) United States
(12) Patent Application Publication
(10) Pub. No.: US 2010/0048671 A1
(43) Pub. Date: Feb. 25, 2010

(54) INHIBITION OF THE 44 KILODALTON ISOFORM OF PIM-1 KINASE RESTORES APOPTOSIS INDUCED BY CHEMOTHERAPEUTIC DRUGS IN CANCER CELLS

(76) Inventors: Yun Qiu, Ellicott City, MD (US); Kexin Xu, Baltimore, MD (US); Yingqiu Xie, Timonium, MD (US)

Correspondence Address: DITTHAVONG MORI & STEINER, P.C., 918 Prince Street, Alexandria, VA, 22314, US

(21) Appl. No.: 11/914,389

(22) PCT Filed: May 12, 2006
(86) PCT No.: PCT/US2006/018483

§ 371 Date: Nov. 14, 2007

Related U.S. Application Data
(60) Provisional application No. 60/681,219, filed on May 14, 2005.

(51) Int. Cl. A61K 31/7105; C07H 21/02; C12N 15/11

(52) U.S. Cl. 514/44 A; 536/24.5; 435/463

(57) ABSTRACT

The present invention relates to a newly discovered 44 kD isoform of Pim-1 kinase made in human cells, and to the gene and messenger RNA for the 44 kilodalton isoform. The invention further describes methods and compounds for treating, especially prostate and hematopoietic cancer, by inhibiting expression of the 44 kD isoform of Pim-1 kinase, or its ability to phosphorylate Etk kinase and breast cancer resistance protein (BCRP).

### CROSS-REFERENCE TO RELATED APPLICATIONS

This application claims benefit of Provisional Appln. 60/681,219, filed May 14, 2005, the entire contents of which are hereby incorporated by reference as if fully set forth herein, under 35 U.S.C. §119(e).

### STATEMENT OF GOVERNMENTAL INTEREST

This invention was made with Government support under NIH Grant: CA85380 and Department of Defense Grant: DAMD17-03-0017. The Government has certain rights in the invention.

### BACKGROUND OF THE INVENTION

1. Field of the Invention

The present invention relates to methods and compounds for treating and preventing cancer, especially prostate and hematopoietic cancer, by inhibiting expression of the 44 kD isoform of Pim-1 kinase, or its ability to bind to Etk kinase and breast cancer resistance protein (BCRP).

2. Description of the Related Art

The serine/threonine kinase Pim-1 was originally identified as a frequently activated cellular proto-oncogene by retrovirus insertion in mice (1). It has also been implicated in the development of hematopoietic and prostatic malignancies. Human Pim-1 gene is mapped to the fragile chromosomal site 6p21 where aberrant translocations were reported in acute nonlymphocytic leukemia (2). The levels of Pim-1 mRNA are often elevated in human leukemias (3). Transgenic mice over-expressing the Pim-1 gene are tumor-prone and susceptible to carcinogens and other tumor promoters, showing a direct role of Pim-1 kinase in oncogenesis (4-5). Pim-1 has been implicated in regulation of the cell cycle and transcription by phosphorylating a number of substrates such as cdc25A, HP1 and p100 (6-9). Increasing evidence suggests that Pim-1 may play a role in regulation of the survival signaling by phosphorylating BAD=BCL2 an antagonist of cell death. (10-11).

Recently, Pim-1 has emerged as a potential diagnostic marker in prostate cancer (12). It has been shown that Pim-1 is frequently upregulated in human prostate cancers as well as in prostate tumor tissues from various mouse models (13-15), showing a potential role of Pim-1 in prostate cancer development and progression. However, very little is known about the function of Pim-1 in prostate cancer cells.

Prostate cancer is the second leading cause of cancer death among men in western countries. Patients with advanced prostate cancer initially benefit from androgen ablation therapy which leads to temporary remission of the tumor due to apoptosis of androgen-sensitive tumor cells. However, the recurrence of androgen-independent tumors is inevitable for most patients and renders the conventional hormone therapy ineffective. Unfortunately, prostate cancer also is often resistant to apoptosis induced by chemotherapeutic agents. Therefore there is still a great need for new methods for treating and preventing prostate cancer.

The past approaches described in this section could be pursued, but are not necessarily approaches that have been previously conceived or pursued. Therefore, unless otherwise indicated herein, the approaches described in this section are not to be considered prior art to the claims in this application merely due to the presence of these approaches in this background section.

### SUMMARY OF THE INVENTION

One Certain set of embodiments is directed to the 44 kilodalton isoform of PIM-1 kinase set forth in SEQ ID NO. 4, or functional equivalent or variant thereof. Another set of embodiments are directed to antisense nucleic acids that are sufficiently complementary to DNA encoding the 44 kilodalton isoform of PIM-1 kinase identified in SEQ ID NO. 1 or to the mRNA identified in SEQ ID 2, to permit specific hybridization under physiologic conditions, and which antisense nucleic acid inhibits expression of the 44 kilodalton isoform. Such antisense nucleic acids can be DNA or RNA or hybrids thereof. In certain set of embodiments the antisense nucleic acid is from about 8 to about 50 nucleobases in length. Other set of embodiments are directed to methods of treating or preventing cancer in an animal, by administering a therapeutic or prophylactic amount of these antisense nucleic acid to inhibit expression of the 44 kilodalton isoform. Expression of the 44 kilodalton isoform of PIM-1 kinase can also be blocked with short interfering RNA that is from about 8 to about 30 nucleobases in length and is complementary to a portion of the mRNA encoding the 44 kilodalton isoform. Such short interfering RNA can be used therapeutically to treat cancer by blocking expression of the 44 kilodalton isoform. Any cancer can be treated using the antisense nucleic acids of the present invention, especially hematopoietic and prostate cancers.

Other set of embodiments of the invention are directed to an expression vector carrying a gene encoding an antisense nucleic acid which antisense nucleic acid is sufficiently complementary to DNA encoding the 44 kilodalton isoform identified in SEQ ID NO. 1 to permit specific hybridization under physiologic conditions, and which antisense nucleic acid inhibits Pim-1 expression, and to a host cell or organism transformed or transfected with such an expression vector. Certain set of embodiments are directed to a transgenic non-human organism comprising a transgene capable of expressing the 44 kilodalton isoform identified by SEQ ID NO. 1, or a functional equivalent thereof.

Certain embodiments are directed to a method of inhibiting the expression of the 44 kilodalton isoform in cells or tissues, by contacting the cells or tissues in vitro with one or more antisense nucleic acids that are sufficiently complementary to DNA encoding the 44 kilodalton isoform identified in SEQ ID NO. 1 or to messenger RNA identified in SEQ ID NO. 2 encoding the 44 kilodalton isoform to permit specific hybridization under physiologic conditions, and which antisense nucleic acid inhibits expression of the 44 kilodalton isoform.

Some set of embodiments of the invention are directed to methods for increasing drug-induced apoptosis in cancer cells in an animal, by administering an antisense nucleic acid that is sufficiently complementary to DNA encoding the 44 kilodalton isoform identified in SEQ ID NO. 1 or to mRNA for the 44 kD isoform to permit specific hybridization under physiologic conditions, in an amount that inhibits expression of the 44 kilodalton isoform. Short interfering RNA can also be used to inhibit expression of the 44 kilodalton isoform thereby increasing drug-induced apoptosis in cancer cells in an animal.

Other set of embodiments are directed to a phosphopeptide that binds to the 44 kilodalton isoform thereby preventing the isoform from phosphorylating Etk or an ABC transporter selected from the group comprising BCRP, ABCG4, ABCG1, MDR1 or ABCA1. Such phosphopeptides can be used to treat or prevent cancer.

Certain set of embodiments of the invention are directed to various screening assays. One set of embodiments is directed to isolated, substantially purified 44 kilodalton isoform of Pim-1 kinase, used in a screening system to identify compounds that bind to the isoform thereby preventing it from phosphorylating or Etk or an ABC transporter selected from the group comprising BCRP, ABCG4, ABCG1, MDR1 or ABCA1. Another set of embodiments of the invention is a screening system for detecting compounds that bind to DNA identified by SEQ ID No. 1 encoding the 44 kilodalton isoform thereby inhibiting expression of 44 kilodalton isoform of Pim-1 kinase, which system includes the DNA identified by SEQ ID NO. 1 or a biologically active fragment or variant thereof. Another set of embodiments is a screening system for detecting compounds that bind to messenger RNA identified by SEQ ID No. 2 encoding the 44 kilodalton isoform thereby inhibiting expression of 44 kilodalton isoform of Pim-1 kinase, which system includes the messenger RNA identified by SEQ ID NO. 2 or a biologically active fragment or variant thereof. Another set of embodiments is a screening system for detecting compounds that bind to the 44 kilodalton isoform or a variant or functional equivalent thereof thereby inhibiting the ability of the isoform to phosphorylate Etk or an ABC transporter selected from the group comprising BCRP, ABCG4, ABCG1, MDR1 or ABCA1, which system includes the 44 kilodalton isoform or a functional equivalent or variant thereof. This system can optionally include Etk or an ABC transporter selected from the group comprising BCRP, ABCG4, ABCG1, MDR1 or ABCA1. In certain set of embodiments of the various screening systems the DNA or mRNA is coupled to a reporter system and is a marker for compounds exhibiting regulating properties of expression of the DNA, such as a fluorescent reporter molecule.

Certain other set of embodiments are directed to a method for treating or preventing cancer in an animal, by administering a therapeutic or prophylactic amount of an antibody or antibody fragment that binds to an ABC transporter selected from the group comprising BCRP, ABCG4, ABCG1, MDR1 or ABCA1, thereby preventing phosphorylation of the ABC transporter by the 44 kilodalton isoform of Pim-1 kinase. For the purpose of this invention and the claimed embodiments, “treating” cancer includes preventing cancer in a precancerous cell.

Other sets of embodiments include an isolated nucleic acid including a sequence that hybridizes under highly stringent conditions to a hybridization probe the nucleotide sequence of which comprises SEQ ID NO. 1 or a biologically active fragment or variant thereof, or the complement of SEQ ID NO. 1; or to a hybridization probe the nucleotide sequence of which consists of SEQ ID NO. 2 or a biologically active fragment or variant thereof, or the complement of SEQ ID NO. 2.

Another set of embodiments is directed to isolated nucleic acids that include a sequence that encodes a polypeptide at least 70% identical to SEQ ID NO. 4, wherein the polypeptide activates Etk. Also described are isolated nucleic acids that include a sequence that encodes a polypeptide comprising the sequence of SEQ ID NO. 4, with up to 50 conservative amino acid substitutions, wherein the polypeptide activates Etk.

Another set of embodiments is directed to an isolated nucleic acid including a sequence that encodes a polypeptide comprising the amino acid sequence of SEQ ID NO. 4, or of an immunogenic fragment of SEQ ID NO. 4 at least 8 residues in length. Some embodiments describe a method of decreasing expression of the 44 kilodalton isoform of Pim-1 kinase in a human cancer cell, by providing an antisense oligonucleotide that inhibits the endogenous expression of the 44 kilodalton isoform of Pim-1 kinase in a human cell; providing the human cancer cell comprising an mRNA encoding the 44 kilodalton isoform of Pim-1 kinase; and introducing the antisense oligonucleotide into the cell, wherein the oligonucleotide decreases expression of the 44 kilodalton isoform of Pim-1 kinase.

### BRIEF DESCRIPTION OF THE DRAWINGS

The present invention is illustrated by way of example, and not by way of limitation, in the figures of the accompanying drawings and in which:

FIG. 1 shows the expression of the two isoforms of Pim-1 in human prostate cancer cells. FIG. 1A: Induction of Pim-1 expression by IL-6 in LNCaP cells. FIG. 1B: The effects of neutralizing antibody against IL-6 on the protein level of Pim-1 in PC3 cells. FIG. 1C: Expression of the two isoforms of Pim-1 by the EST clones in transfected LNCaP, 22Rv1 and PC3 cells FIG. 1D: The N-terminal unique sequence in human 44 kD Pim-1. Part of the sequence of the 5′ end of Pim-1 cDNA in the EST clones is shown. The putative alternative translation initiation site CUG and the start codon AUG are in boldface. FIG. 1E: Alignment of the unique N-terminal sequence in the 44 kD Pim-1 from human and mouse origins. Proline-rich motifs are underlined and bold. The translation initiation sites of the two isoforms are shown by arrows.

FIG. 2 shows the expression of the 44 kD Pim-1 isoform in prostate cancer cells. FIG. 2A: Specificity of the anti-mouse Pim-1L rabbit polyclonal antibody determined by immunoblotting analysis on the total cell lysates from cells transfected with Flag-tagged Pim-1L or Pim-1S. The anti-Flag antibody served as a positive control. FIG. 2B: Expression of Pim-1L in total cell lysates of human prostate cancer cell lines CWR-R1 (R1), LNCaP, PC3 and 22Rv1. Actin is a control. FIG. 2C: Tissue array showing that expression of Pim-1L is upregulated in human prostate tumor compared to benign prostate hyperplasia subjected to immunohistochemical staining using anti-Pim-1L.

FIG. 3 shows the subcellular localization of Pim-1L and Pim-1 S in prostate cancer cells. FIG. 3A: shows the distinct localization of Pim-1L and Pim-1 S in LNCaP cells transfected with Flag-tagged Pim-1L or Pim-1S using confocal imaging FIG. 3B: Co-localization of Pim-1L and Etk in LNCaP cells transfected with Flag-tagged Pim-1L and T7-Etk at 48 hours post-transfection.

FIG. 4 shows that the interaction between Pim-1L and tyrosine kinase Etk activates Etk kinase activity. FIG. 4A: COS-1 or 22Rv1 cell lysates transfected with the plasmids indicated were subjected to immuno-precipitation with the anti-Etk antibody followed by immunoblotting with anti-Flag antibody. The effects of Pim-1 on Etk kinase activity were examined by immunoprecipitation with anti-Etk and followed by immunoblotting with anti-phosphotyrosine (αpY). FIG. 4B: shows that the SH3 domain of Etk is required for binding to Pim-1L. T7-tagged Etk or Etk mutants co-transfected with Flag-Pim-1L into COS-1 cells were immunoprecipitated with polyclonal anti-Etk antibody and then immunoblotted with anti-Flag antibody. Immunoblots of total cell lysates with anti-Flag and anti-T7 were included to monitor the protein levels of Pim1 and Etk in the total cell lysates. FIG. 4C: shows the direct interaction between the SH3 domain of Etk and the proline-rich region of Pim-1L using immobilized GST-SH3(Etk) fusion protein or the GST control incubated with 293T cell lysates that over-express Flag-Pim-1L, Pim-1LΔP or Pim-1LPA mutants. The associated proteins were resolved by the SDS-PAGE and detected by immunoblotting with anti-Flag antibody (Top). The same blot was stained with Coomassie Blue to monitor the amount of GST-SH3 fusion protein bound to the glutathione beads (Bottom). FIG. 4D: shows that endogenous Etk is associated with Pim-1L in lysates from LNCaP and PC3 prostate cancer cells immunoprecipitated with polyclonal Pim-1 antibody or preimmune serum control that was immunoblotted with monoclonal anti-Etk antibody or anti-Pim-1 antibodies. The total cell lysates (TCL) serve as the positive controls. FIG. 4E: shows the effects of Pim-1 on endogenous Etk kinase activity. HUVEC (human umbilical vein endothelial cells) or LNCaP cells infected with lenti-virus encoding either Flag-Pim 1L, Pim-1LΔP or vector alone were immunoprecipitated using anti-Etk. Tyrosine phosphorylation of Etk and Stat3 was determined by immunoblotting with anti-phosphotyrosine (4G10) and anti-phospho Stat3 Y705, respectively. The expression of Flag-Pim1L or Pim-1LΔP produced by the respective lenti-virus in COS-1 cells was determined by immunoblotting with anti-Flag antibody. FIG. 4F: shows the effects of Pim-1 on Etk kinase activity in COS-1 cells transfected with the indicated plasmids, immunoprecipitated with anti-Etk and assayed using the in vitro kinase assay (IVK) carried out by using GST-Gab1 as a substrate. (top) The Pim-1 expression level in the transfected cells was determined by immunoblotting total cell lysates with anti-Flag (bottom).

FIG. 5: shows that over-expression of Pim-1L in LNCaP cells confers drug resistance. FIG. 5A: LNCaP cells infected with lenti-virus encoding Flag-Pim-1S, Pim-1L, Pim-1LKM (kinase-deficient mutant) or vector control were treated with doxorubicin (0.1 ug/ml) for the times as indicated. Cell viability was determined by the WST-1 assay. The data were expressed as the mean of triplicates of the doxorubicin-treated samples relative to the untreated controls. * p<0.01 compared with the vector control. The expression of the Pim-1 proteins encoded by the lenti-viruses was determined by immunoblotting with anti-Flag antibody. FIG. 5B: LNCaP cells were infected with the lenti-viruses encoding Flag-Pim-IL and the kinase-inactive T7-EtkKQ simultaneously. The response to doxorubicin was determined at 20 hour as above. * p<0.01 compared with the vector control; ** p<0.01 compared with Pim-1L alone. Expression of Flag-Pim-1L and T7-EtkKQ was monitored by immunoblotting with anti-Flag and anti-T7 antibody, respectively. FIG. 5C: LNCaP cells infected with the lenti-viruses encoding the indicated proteins as in 5A were treated with 10-100 nM mitoxantrone for 48 h; cell viability was determined as in 5A. * p<0.01 compared with the vector control. FIG. 5D: Etk mediates Pim-1L-induced resistance to mitoxantrone. LNCaP cell were infected with the lenti-viruses and the mitoxatrone response was determined. * p<0.01 compared with the vector control; ** p<0.01 compared with Pim-1L alone. FIG. 5E: shows that Pim-1L disrupts the interaction between Etk and p53. LNCaP cells were transfected with the plasmids as indicated. The association of Etk with p53 was determined by immunoprecipitation with anti-p53 and followed by immunoblotting with anti-T7 antibody. The expression of p53, Etk or Pim-1 in these cells was monitored by immunoblotting with anti-p53, anti-T7 and anti-Flag antibody, respectively.

FIG. 6 shows immunofluorescence staining of endogenous BCRP in prostate cancer cells. CWR-R1 and 22Rv1 cells were grown on cover slips, fixed and then subjected immunostaining with a monoclonal anti-BCRP antibody (clone BXP-21, Chemicon).

FIG. 7 Association of BCRP and Pim-1L in mammalian cells. FIG. 7A: Co-immunoprecipitation of over-expressed BCRP and Pim-1L. 293T cells were co-transfected with plasmids encoding HA-tagged BCRP with vector control or with Flag-tagged Pim-1L or Pim-1S. Cells were lysed at 48 h post-transfection, and subjected to immunoprecipitation with monoclonal anti-Flag antibody, followed by immunoblotting with anti-HA. The expression of HA-tagged BCRP and Flag-tagged Pim-1L and Pim-1 S was monitored by immunoblotting with anti-HA and anti-Flag respectively. FIG. 7B: co-localization of ectopically expressed Flag-tagged Pim-1L and HA-tagged BCRP in prostate cancer LNCaP cells. LNCaP cells transiently transfected with Flag-tagged Pim-1L and HA-tagged BCRP. Cells were fixed at 48h posttransfection and subjected to immunostaining with a monoclonal anti-HA antibody and a polyclonal anti-Pim-1L.

FIG. 8 Detection of threonine and tyrosine phosphorylation of endogenous BCRP in breast MCF7-MX and prostate CWR-R1 cancer cell lysates immunoprecipitated with monoclonal BCRP antibody or mouse IgG control, followed by immunoblotting with anti-phosphothreonine (αpThr) and anti-BCRP, respectively.

FIG. 9 shows that Pim-1L induces endogenous BCRP threonine phosphorylation in the MCF7-MX resistant cell line. MCF7-MX cells were infected with lenti-virus encoding vector control and Pim-1L, or Pim-1L kinase dead mutant (Pim-1LKM). Cell lysates were subjected to immunoprecipitation with monoclonal anti-BCRP antibody, followed by immunoblotting with anti-phosphothreonine (αpThr).

FIG. 10 shows that inhibition of Pim-1 expression reduces threonine phosphorylation of the endogenous BCRP in MCF7-MX breast cancer cell lines. MCF7-MX cell lysates infected with lenti-virus encoding Vector control and Pim-1 SiRNA(SiPim-1) were subjected to immunoprecipitation with monoclonal BCRP antibody, followed by immunoblotting with anti-phosphothreonine (αpThr).

FIG. 11 shows that inhibition of Pim-1 expression increases drug sensitivity in MCF7-MX cells. FIG. 11A: MCF7-MX cells infected with lenti-virus encoding vector control and Pim-1 SiRNA were treated with Flavopiridol (15 μM) or DTX (50 μM) as indicated for 2 days. Cell viability was determined using the WST-1 assay. P<0.05 FIG. 11B: expression levels of Pim-1, BCRP and actin analyzed by immunoblotting with the respective antibodies.

FIG. 12 shows that inhibition of Pim-1 expression increases drug sensitivity in CWR-R1 cells. FIG. 12A: CWR-R1 cells infected with lenti-virus encoding vector control and Pim-1 SiRNA were treated with MX (2 μM), TPT (5 μM) or DTX (50 nM), respectively for 2 days. Cell viability was determined using the WST-1 assay. FIG. 12B: immunoprecipitation performed by using anti-BCRP.

FIG. 13 shows that downregulating Pim-1 sensitizes LNCaP cells over-expressing BCRP to chemotherapeutic drugs. FIG. 13A: LNCaP cells infected with lenti-virus encoding vector control, BCRP, Pim-1SiRNA or both were treated with the indicated drugs MX (0.5 μM) or TPT (0.1 μM) for 2 days. Cell viability was determined using the WST-1 assay FIG. 13B: Immunoprecipitation was performed by using anti-BCRP.

FIG. 14A: Schematic structure of human BCRP. FIG. 14B: shows that the threonine phosphorylation site (T362) is conserved in several members of ABC transporters.

FIG. 15 shows the effect of phosphorylation of T362 of BCRP on BCRP-mediated resistance to apoptosis induced by a three day exposure to chemotherapeutic drugs mitoxantrone (FIG. 15A), docetaxel (FIG. 15B), and topotecan (FIG. 15C) in LNCaP cells infected with the lenti-virus encoding the HA-tagged BCRP or the T362A mutant. Cell viability was determined using the WST-1 assay. FIG. 15D: expression of HA-tagged BCRP or the T362A mutant examined by immunoblotting with anti-HA.

FIG. 16 shows that Pim-1L induces BCRP phosphorylation at T362 in 293T cells. 293T cell lysates transfected with the plasmids indicated were examined by immunoprecipitation with anti-BCRP, followed by immunoblotting with anti-phosphothreonine to determine the effects of Pim-1 on BCRP phosphorylation.

FIG. 17 shows that Pim-1L increases BCRP-mediated drug resistance in LNCaP cells through phosphorylation of T362. FIG. 17A: At 72 hour post-infection LNCaP cells infected with the lenti-virus encoding the HA-tagged BCRP, or the T362A mutant with the kinase active Pim-1L, or the kinase-inactive Pim-1LKM were treated with 1 μM mitoxantrone (MT) or 2 μM Toptecan (TPT) for 2 days. Cell viability was determined using the WST-1 assay; expression of HA-tagged BCRP (or BCRP T362A mutant) and Flag-tagged Pim-1L (or Pim-1LKM mutant) was monitored by immunoblotting with anti-HA and anti-Flag antibodies respectively (Bottom panels).

FIG. 18 shows that BCRP dimerization by Pim-1L depends on phosphorylation of T362. Lysates of 293T cells transfected with the plasmids indicated were examined by immunoprecipitation with anti-HA and followed by immunoblotting with anti-Myc.

FIG. 19 shows that Pim-1 L, Etk and BCRP are upregulated in the docetaxel (DTX)-resistant LNCaP cell line. FIG. 19A: Total cell lysates of the DTX-resistant LNCaP derivative cell line were subjected to immunoblotting with anti-BCRP, Pim-1, Etk, or MDR1 as indicated. FIG. 19B: Pim-1L is upregulated in mitoxantrone-resistant breast cancer cells. Western Blot was performed to detect the level of Pim-1 expression in MCF7/MX and the parental drug-sensitive MCF7/MX cells using a monoclonal anti-Pim-1 antibody (clone 19F7, Santa Cruze). 293T cells transfected with the EST clone expressing both Pim-1L and Pim-1S was used as a positive control.

### DETAILED DESCRIPTION

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in inhibit diagram isoform in order to avoid unnecessarily obscuring the present invention.

It has been discovered that human cells express a 44 kD isoform of Pim-1 kinase (hereafter the “44 kD isoforms” or “Pim-1L” are used interchangeably), and we have isolated and sequenced the gene for the this protein. One embodiment is directed to the 44 kD isoform having the amino acid sequence set forth in SEQ ID NO. 4, or functional equivalent or variant thereof. An embodiment is also directed to the gene identified by SEQ ID NO. 1 encoding the mRNA for the 44 kD isoform, as well as the mRNA for the 44 kD isoform identified in SEQ ID NO. 2, or a biologically active form or variant thereof.

It has been discovered that the 44 kD isoform of Pim-1 kinase phosphorylates and thereby activates Etk, especially in cancer cells. This is activation prevents Etk from binding to p53, which makes the cancer cells resistant to drug induced apoptosis. Thus, methods and compositions are described for treating or preventing cancer, especially prostate cancer or hematopoietic cancers, by administering isolated nucleic acids that inhibit expression of the 44 kD isoform in the cancer cell. All of the nucleic acids of the present invention are isolated nucleic acids.

As used herein, the term “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand). As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that flank a Pim-1L gene). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome. It shall be understood that the nucleic acids of the present invention (including genes, mRNA, cDNA, antisense and short interfering RNA) are isolated nucleic acids, even though the term “isolated” is not included every time a nucleic acid of the invention is discussed.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lenti-virus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

In accordance with the present invention, a defined nucleic acid includes not only the identical nucleic acid but also any minor base variations including in particular, substitutions in cases which result in a synonymous codon (a different codon specifying the same amino acid residue) due to the degenerate code in conservative amino acid substitutions. Sequence variants can be found in coding and non-coding regions, including exons, introns, promoters, and untranslated sequences.

The term “nucleic acid sequence” also includes the complementary sequence to any single stranded sequence given regarding base variations. As used herein with respect to nucleic acids “isolated” means any of a) amplified in vitro by, for example, polymerase chain reaction (PCR), b) recombinantly produced by cloning, c) purified by, for example, gel separation, or d) synthesized, such as by chemical synthesis.

In the context of this invention, the term “oligonucleotide” refers to an oligomer or polymer of ribonucleic acid (RNA) or deoxyribonucleic acid (DNA) or mimetics thereof. Variants of the nucleic acids of the present invention include, inter alia, oligonucleotide forms thereof. This term includes nucleic acids composed of naturally-occurring nucleobases, sugars and covalent internucleoside (backbone) linkages as well as nucleic acids having non-naturally-occurring portions which function similarly. Such modified or substituted nucleic acids are often preferred over native forms because of desirable properties such as, for example, enhanced cellular uptake, enhanced affinity for nucleic acid target and increased stability in the presence of nucleases. The term “nucleic acids” therefore includes oligonucleotides.

Certain embodiments are directed to isolated antisense nucleic acids that are sufficiently complementary to the gene (DNA) encoding the 44 kilodalton (kD) isoform of human PIM-1 kinase (Pim-1L) identified in SEQ ID NO. 1, or to messenger RNA encoding the 44 kilodalton (kD) isoform of human PIM-1 kinase (Pim-1L) identified in SEQ ID NO. 2, to permit specific hybridization under physiologic conditions, and which antisense nucleic acids inhibit expression of the 44 kilodalton isoform. Other embodiments are directed to short-interfering RNA that inhibits expression of Pim-1L. “Hybridization” and “complementarity” are discussed in more detail in the Definitions section below.

Other embodiments of the invention are directed to screening systems that identify compounds that bind to the gene encoding Pim-1L (or a biologically active fragment or variant thereof) thereby inhibiting its transcription, and to compounds that bind to mRNA for Pim-1L thereby inhibiting its translation. Such screening systems include the respective gene and/or mRNA for Pim-1L (or biologically active fragment or variant), including fragments that carry the proline-rich regions of the gene and mRNA or variants thereof. The gene and mRNA for Pim-1L can be an isolated and purified product, or recombinant or synthesized nucleic acids made as described below. In another embodiment, Pim-1L protein (or a functional equivalent or variant thereof) is used in a screening system as a marker for compounds that bind to the protein, preferably to a proline-rich region (PXXP region) thereby preventing Pim-1L from phosphorylating or activating Etk. Such compounds are useful in treating or preventing cancer by preventing Pim-1L from activating Etk.

Another embodiment is directed to an expression vector carrying a gene for Pim-1L or a biologically active fragment or variant thereof, which vector can be used to make transgenic cells or animals that over-express Pim-1L. Such animals would over-express Pim-1L and could be used to test compounds that interfere with Pim-1L expression, or binding and activation of Etk. Other embodiments are directed to methods and compounds that increase drug-induced apoptosis in cancer cells in an animal; such compounds include nucleic acids that inhibit expression of Pim-1L at the gene or mRNA level or protein levels.

It has also been discovered that Pim-1L phosphorylation of breast cancer resistance protein (BCRP) causes BCRP-induced chemotherapeutic drug resistance to apoptosis. Therefore certain embodiments are directed to treating or preventing cancer by inhibiting Pim-1L expression or phosphorylation of BCRP by Pim-1L, thus preventing BCRP-induced chemotherapeutic drug resistance.

In the following description, for the purposes of explanation, numerous specific details are set forth in order to provide a thorough understanding of the present invention. It will be apparent, however, to one skilled in the art that the present invention may be practiced without these specific details. Particular embodiments of the invention are descried below in the context of treating prostate cancer, however, the invention is not limited to this context.

Protein kinase Pim-1 has been implicated in the development of hematopoietic and prostatic malignancies. The experiments herein show that two isoforms, the 44 kD and 33 kD Pim-1, are expressed in all human prostate cancer cell lines examined. CWR-R1 cells, COS-1 cells, LNCaP cells, PC3 cells, and 22Rv1 cells were tested. It was known that the murine Pim-1 gene encodes two proteins with molecular weight 34 kD and 44 kD by utilizing two alternative translation initiation sites (17). However it was not known until now that the 44 kD isoform is also expressed in human cells. Translation of the 44 kD Pim-1 (Pim-1L) was reported to initiate at a non-conventional start codon CUG in mouse, while the 33 kD isoform (Pim-1S) is translated from a downstream AUG codon. It has yet to be determined whether regulation of Pim-1 kinase by the alternative translation initiation is conserved in human cells. Our current knowledge about Pim-1 kinase is largely derived from the studies on the 33 kD isoform Pim-1. Several potential substrates for Pim-1, including p100, cdc25A, HP1 and TFAF2/SNX6, have been identified through various genetic screenings (6-9, 18)

Recently, Pim-1 has emerged as a potential diagnostic marker in prostate cancer (12). Pim-1 is frequently upregulated in human prostate cancers as well as in the prostate tumor tissues from various mouse models. However, very little is known about the function of Pim-1 in prostate cancer cells. Recently, we showed that Pim-1 kinase is regulated by interleukin 6 (IL6) in prostate cancer cells and involved in IL6-induced ligand-independent activation of the androgen receptor (19). We also demonstrated that the 33 kD Pim-1 (hereafter “Pim-1 S”) and the Tec family tyrosine kinase Etk can act synergistically to promote the androgen receptor mediated transcriptional activity in prostate cancer cells. Tec family kinases were identified as potential targets for p53 (19) in an SH3 domain array screening for them ligands of the p53 proline-rich domain. Upon treatment of prostate cancer cells with chemotherapeutic drugs, p53 binds to Etk through an interaction between the SH3 domain of Etk and the proline-rich domain of p53, a reaction that inactivates Etk tyrosine kinase. Down-regulation of Etk kinase activity appears to be required for p53-induced apoptosis (19).

### Human Cells Express the 44 kD Isoform of Pim-1 Kinase

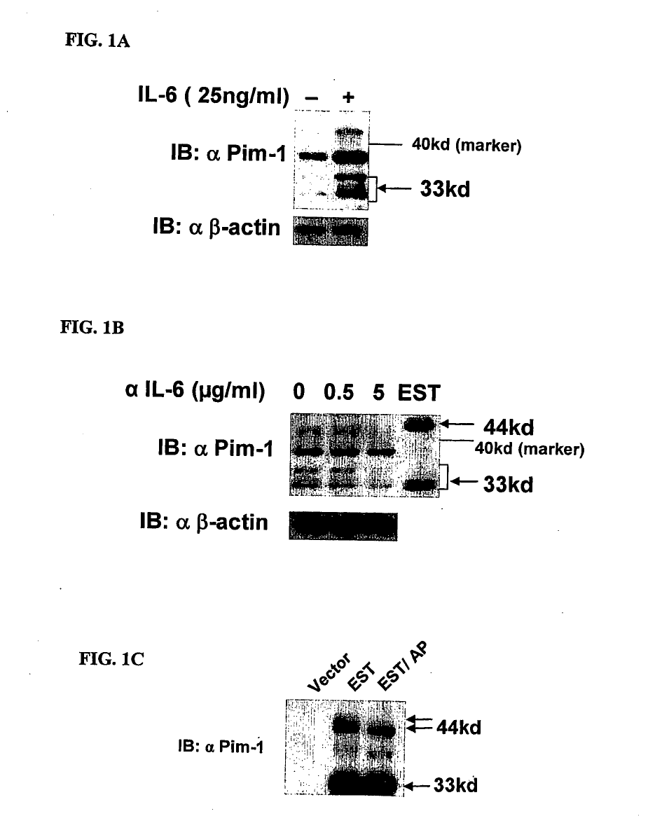

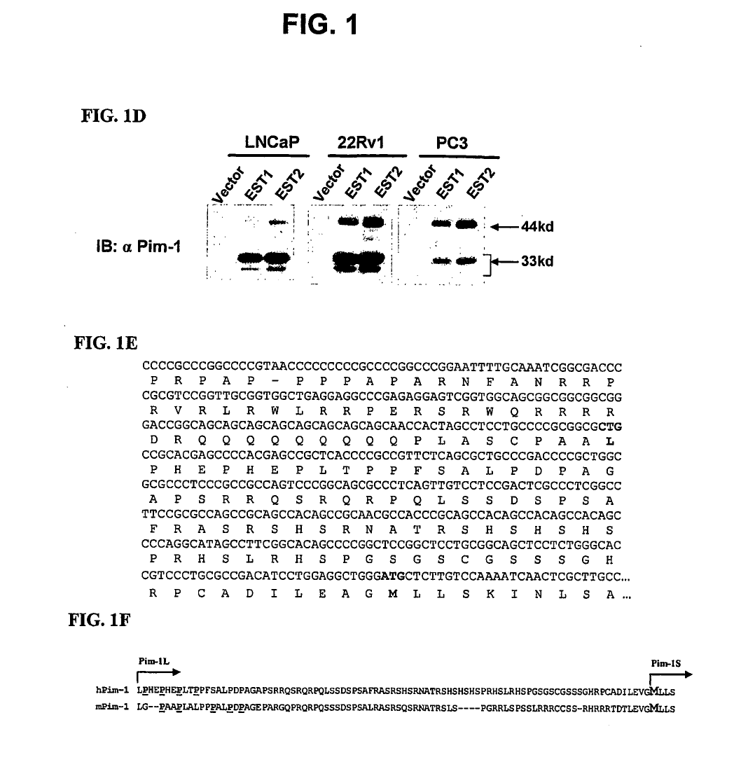

We previously showed that the 33 kD Pim-1 S isoform is up-regulated in IL-6 treated LNCap cells (19). In addition to the doublet bands corresponding to the previously reported p33 kD Pim-1, it has now been discovered that a protein with higher molecular weight around 44 kD is also induced by IL-6 in human prostate cancer cells, and it reacts with the monoclonal anti-Pim-1 antibody (FIG. 1A). Induction of Pim-1 expression by IL-6 was assessed in serum starved LNCaP cells that were treated with 25 ngml IL-6 for 24 hour at 37 degrees. The level of Pim-1 protein was examined by immunoblotting with a monoclonal anti-Pim-1 antibody (Top). The same blot was then probed with anti-actin to monitor the sample loading (Bottom). A similar pattern was observed in human PC3 cells in which an IL-6 autocrine loop is present. The results presented below show that this is the human 44 kD Pim-1L isoform and that it is substantially homologous (about 60%) to the mouse 44 kD isoform. Treatment with a neutralizing anti-IL6 antibody dramatically suppressed the expression of both the 33 kD and 44 kD proteins (FIG. 1B). One embodiment of the present invention is directed to the isolated human 44 kD isoform identified by SEQ ID NO. 4 or a functional equivalent or variant thereof and its use in screening assays. We are the first to isolate and describe the gene for the 44 kD isoforms identified in SEQ ID NO. 1, and the sequence of the mature mRNA for it shown in SEQ ID NO. 2 both of which are claimed as embodiments of the present invention.

Since it was reported that a 44 kD isoform of Pim-1 is encoded in mouse cells as a result of the usage of an alternative start codon (17), we conducted a study to determine whether the 44 kD mouse protein is the 44 kD isoform of Pim-1 in humans. This was done by expressing the Expressed Sequence Tags (EST) clones in which the human Pim-1 cDNA sequence is under the control of the cytomegalovirus (CMV) promoter. As shown in FIG. 1C, two major isoforms of Pim-1 proteins were expressed in three human prostate cancer cell lines transfected with two independent human EST clones in which the cDNA of Pim-1 gene is under the control of the CMV promoter. The expression of Pim-1 proteins was examined by immunoblotting with monoclonal-Pim-1 antibody. In both LNCaP and 22Rv1 cells, the 33 kD Pim-1 (Pim-1S) was the predominant isoform while in PC3 cells, the 44 kD Pim-1L and 33 kD isoforms were expressed at similar levels. Sequencing of these two EST clones revealed the coding sequence of human Pim-1 shares about 60% homology with the mouse 44 kD Pim-1. Like the mouse 44 kD Pim-1, a leucine residue is used as an alternative translation start site (FIG. 1D). FIG. 1E shows the alignment of the unique N-terminal sequence in the 44 kD Pim-1 from human (hPim-1) and mouse (mPim-1) origins. The proline-rich motifs are underlined and bold. The translation initiation sites of two isoforms are shown by arrows.

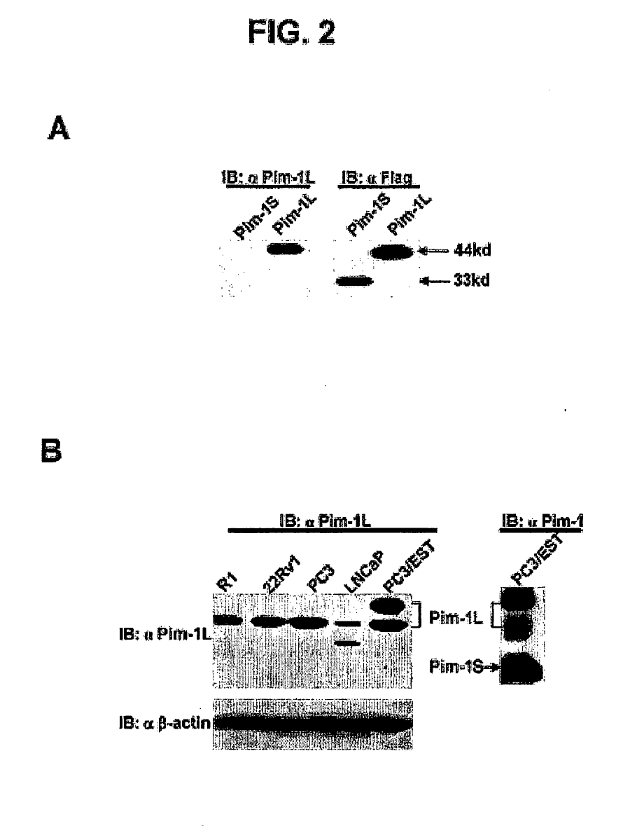

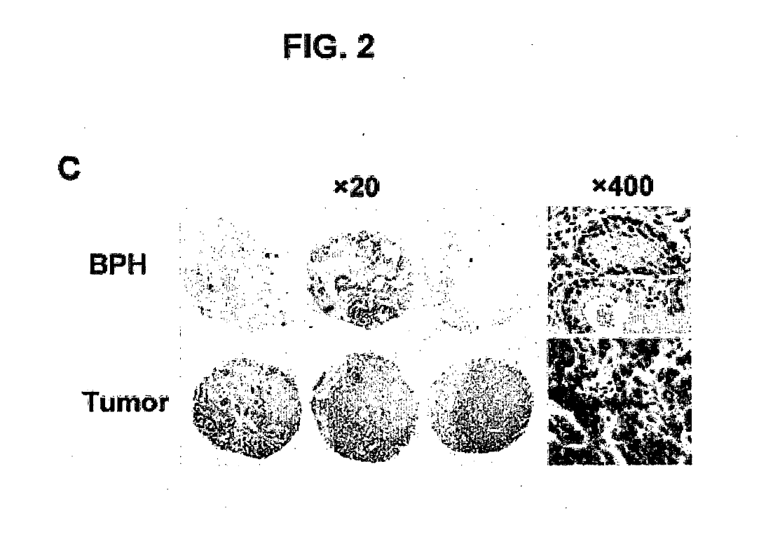

Interestingly, three PXXP motifs are identified in the amino acid sequence for the 44 kD isoform in humans, which are unique to this isoform. It is known that the SH3-domain of Etk is also a proline-rich region. Thus the PXXP motif in the 44 kD Pim-1L isoform could potentially interact with SH3-domain-containing proteins like Etk. To confirm that the 44 kD isoform (Pim-1L) is indeed expressed in prostate cancer cells, a mouse polyclonal antibody (anti-Pim-1L) that specifically recognizes the sequence of the first 91 amino acids which are unique to the 44 kD isoform was developed. FIG. 2A shows that the anti-Pim-1L polyclonal antibody only detects the transfected Flag-tagged 44 kD Pim-1 (Pim-1L) and not the 33 kD isoform (Pim-1 S), because the epitopes recognized by this antibody are missing in Pim-1S. As shown in FIG. 2B, Pim-1L is detectable in all four human prostate cancer cell lines tested. The rabbit polyclonal antibody specific for the 44 kD Pim-1 (Pim-1L) was developed as described in the Examples. The specificity of this antibody was determined by the immunoblotting analysis on the total cell lysates from the cells transfected with Flag-tagged Pim-1L or Pim-1 S. The anti-Flag antibody served as a positive control. To evaluate the expression of Pim-1L in human prostate cancer cell lines, the total cell lysates from CWR-R1 (R1), LNCaP, PC3 and 22Rv1 cells were subjected to immunoblotting with anti-Pim-1L. Anti-actin was used as a loading control. Expression of Pim-1L is upregulated in human prostate tumor tissues as is shown in FIG. 2C. The prostate tissue array was subjected to IHC staining by using anti-Pim-1L as described in the Examples. A representative field of the array was shown.

This study reports the newly discovered and isolated 5000 nucleotide long gene for Pim-1L (the 44 kD isoform). We also sequenced the gene encoding Pim-1L set forth in SEQ ID No. 1, which is the cDNA Gene Bank sequence made from mature mRNA encoding human Pim-1L (without introns or untranslated regions). The sequence for the mRNA is SEQ ID NO. 2. One embodiment of the invention is directed to the gene in SEQ ID NO. 1 or a biologically active form or variant thereof. The first 227 nucleotides of SEQ ID NO. 1 are the untranslated region of the Pim-1L gene. This untranslated region is possibly involved in regulation of gene expression at a posttranslational level, and is nonetheless still a target for short interfering RNA and antisense nucleotides that block transcription. The coding region of the human 44 kD Pim-1 (hPim-1L) gene (the sense strand) is nucleotides 227-1438 of SEQ ID NO. 1. An alternative translational initiation codon CTG (large font, bold, all caps) is used for translation of the 44 kD hPim-1. The sequence encoding the first 91 unique amino acids of the 44 kD Pim-1 is underlined. The ATG start codon for the 33 kD Pim-1 follows the 91 unique amino acids and is bold and all caps. One embodiment is directed to isolated antisense nucleotides including those that are sufficiently complementary to SEQ ID NO. 1 to inhibit transcription of this gene, or a biologically active fragment or variant thereof.

The mRNA sequence for Pim-1L identified in SEQ ID NO. 2 is also an embodiment of the present invention, as are isolated antisense DNA or RNA nucleotides that inhibit translation of the Pim-1L mRNA. Such antisense nucleic acids include those that are sufficiently complementary to SEQ ID NO. 2 to permit specific hybridization under physiologic conditions, thereby blocking translation of the mRNA and expression of Pim-1L.

THE GENE ENCODING MATURE MRNA WITHOUT INTRONS FOR THE 44 KILODALTON ISOFORMS OF PIM-1 KINASE

SEQUENCE ID NO. 1

aaaactgacccccaaccccctaacactcgaagatagggccgtatcactaccccgcccggccccgtaacccccccccgccccggcccggaattttgcaaatcg
gcgaccccgcgtcccggttgcggtggctgaggaggcccgagaggagtcggtggcagcggcggcggcgggaccggcagcagcagcagcagcagcagcagcaac
cactagcctcctgccccgcggcgCTGccgcacgagccccacgagccgctcaccccgccgttctcagcgctgcccgaccccgctggcgcgccctcccgccgcc
agtcccggcagcgccctcagttgtcctccgactcgccctcggccttccgcgccagccgcagccacagccgcaacgccacccgcagccacagccacagccaca
gccccaggcatagccttcggcacagccccggctccggctcctgcggcagctcctctgggcaccgtccctgcgccgacatcctggaggttggg [symbol] ctcttg
tccaaaatcaactcgcttgcccacctgcgcgccgcgccctgcaacgacctgcacgccaccaagctggcgcccggcaaggagaaggagcccctggagtcgcag
taccaggtgggcccgctactgggcagcggcggcttcggctcggctactcaggcatccgcgtctccgacaacttgccggtggccatcaaacacgtggagaagg
accggatttccgactggggagagctgcctaatggcactcgagtgcccatggaagtggtcctgctgaagaaggtgagctcgggtttctccggcgtcattaggc
tcctggactggttcgagaggcccgacagtttcgtcctgatcctggagaggnccgagccggtgcaagatctcttcgacttcatcacggaaaggggagccctgc
aagaggagctggcccgcagcttcttctggcaggtgctggaggccgtgcggcactgccacaactgcggggtgctccaccgcgacatcaaggacgaaaacatcc
ttatcgacctcaatcgcggcgagctcaagctcatcgacttcgggtcgggggcgctgctcaaggacaccgtctacacggacttcgatgggacccgagtgtata
gccctccagagtggatccgctaccatcgctaccatggcaggtcggcggcagctggtccctggggatcctgctgtatgatatggtgtgtggagatattccttt
cgagcatgacgaagagatcatcaggggccaggttttcttcaggcagagggtctcttcagaatgtcagcatctcattagatggtgcttggccctgagaccatc
agataggccaaccttcgaagaaatccagaaccatccatggatgcaagatgttctcctgccccaggaaactgctgagatccacctccacagcctgtcgccggg
gcccagcaaatagcagcctttctggcaggtcctcccctctcttgtcagatgcccgagggaggggaagcttctgtctccagcttcccgagtaccagtgacacg
tctcgccaagcaggacagtgcttgatacaggaacaacatttacaactcattccagatcccaggcccctggaggctgcctcccaacagtggggaagagtgact
ctccaggggtcctaggcctcaactcctcccatagatactctcttcttctcataggtgtccagcattgctggactctgaaatatcccgggggtggggggtggg
ggtgggtcagaaccctgccatggaactgttttcttcatcatgagttctgctgaatgccgcgatgggtcaggtaggggggaaacaggttgggatgggatagga
ctagcaccattttaagtccctgtcacctcttccgactctttctgagtgccttctgtggggactccggctgtgctgggagaaatacttgaacttgcctctttt
acctgctgcttctccaaaaatctgcctgggttttgttccctatttttctctcctgtcctccctcaccccctccttcatatgaaaggtgccatggaagaggct
acagggccaaacgctgagccacctgcccttttttctgcctcctttagtaaaactccgagtgaactggtcttcctttttggtttttacttaactgtttcaaag
ccaagacctcacacacacaaaaaatgcacaaacaatgcaatcaacagaaaagctgtaaatgtgtgtacagttggcatggtagtatacaaaaagattgtagtg
gatctaatttttaagaaattttgcctttaagttattttacctgtttttgtttcttgttttgaaagatgcgcattctaacctggaggtcaatgttatgtattt
atttatttatttatttggttcccttcctattccaagcttccatagctgctgccctagttttctttcctcctttcctcctctgacttggggaccttttggggg
agggctgcgacgcttgctctgtttgtggggtgacgggactcaggcgggacagtgctgcagctccctggcttctgtggggcccctcacctacttacccaggtg
ggtcccggctctgtgggtgatggggaggggcattgctgactgtgtatataggataattatgaaaagcagttctggatggtgtgccttccagatcctctctgg
ggctgtgttttgagcagcaggtagcctgctggttttatctgagtgaaatactgtacaggggaataaaagagatcttatttttttttttatacttggcgtttt
ttgaataaaaaccttttgtcttaaaaaaaaaaaaaaaaaaaaaaaaaaaaaaaaaaaaaaaaaaaa
MESSENGER RNA FOR THE PIM-1L GENE

SEQ ID NO. 2

aaaacugacccccaacccccuaacacucgaagauagggccguaucacuaccccgcccggccccguaacccccccccgccccggcccggaauuuugcaaaucg
gcgaccccgcguccgguugcgguggcugaggaggcccgagaggagucgguggcagcggcggcggcgggaccggcagcagcagcagcagcagcagcagcaacc
acuagccuccugccccgcggcgCUGccgcacgagccccacgagccgcucaccccgccguucucagcgcugcccgaccccgcuggcgcgcccucccgccgcca
gucccggcagcgcccucaguuguccuccgacucgcccucggccuuccgcgccagccgcagccacagccgcaacgccacccgcagccacagccacagccacag
ccccaggcauagccuucggcacagccccggcuccggcuccugcggcagcuccucugggcaccgucccugcgccgacauccuggagguuggg [symbol] cucuugu
ccaaaaucaacucgcuugcccaccugcgcgccgcgcccugcaacgaccugcacgccaccaagcuggcgcccggcaaggagaaggagccccuggagucgcagu
accaggugggcccgcuacugggcagcggcggeuucggcucggucuacucaggcauccgcgucuccgacaacuugccgguggccaucaaacacguggagaagg
accggauuuccgacuggggagagcugccuaauggcacucgagugcccauggaagugguccugcugaagaaggugagcucggguuucuccggcgucauuaggc
uccuggacugguucgagaggcccgacaguuucguccugauccuggagaggnccgagccggugcaagaucucuucgacuucaucacggaaaggggagcccugc
aagaggagcuggcccgcagcuucuucuggcaggugcuggaggccgugcggcacugccacaacugcggggugcuccaccgcgacaucaaggacgaaaacaucc
uuaucgaccucaaucgcggcgagcucaagcucaucgacuucgggucgggggcgcugcucaaggacaccgucuacacggacuucgaugggacccgaguguaua
gcccuccagaguggauccgcuaccaucgcuaccauggcaggucggcggcagucuggucccuggggauccugcuguaugauaugguguguggagauauuccuu
ucgagcaugacgaagagaucaucaggggccagguuuucuucaggcagagggucucuucagaaugucagcaucucauuagauggugcuuggcccugagaccau
cagauaggccaaccuucgaagaaauccagaaccauccauggaugcaagauguucuccugccccaggaaacugcugagauccaccuccacagccugucgccgg
ggcccagcaaauagcagccuuucuggcagguccuccccucucuugucagaugcccgagggaggggaagcuucugucuccagcuucccgaguaccagugacac
gucucgccaagcaggacagugcuugauacaggaacaacauuuacaacucauuccagaucccaggccccuggaggcugccucccaacaguggggaagagugac
ucuccagggguccuaggccucaacuccucccauagauacucucuucuucucauagguguccagcauugcuggacucugaaauaucccggggguggggggugg
gggugggucagaacccugccauggaacuguuuucuucaucaugaguucugcugaaugccgcgaugggucagguaggggggaaacagguugggaugggauagg
acuagcaccauuuuaagucccugucaccucuuccgacucuuucugagugccuucuguggggacuccggcugugcugggagaaauacuugaacuugccucuuu
uaccugcugcuucuccaaaaaucugccuggguuuuguucccuauuuuucucuccuguccucccucacccccuccuucauaugaaaggugccauggaagaggc
uacagggccaaacgcugagccaccugcccuuuuuucugccuccuuuaguaaaacuccgagugaacuggucuuccuuuuugguuuuuacuuaacuguuucaaa
gccaagaccucacacacacaaaaaaugcacaaacaaugcaaucaacagaaaagcuguaaauguguguacaguuggcaugguaguauacaaaaagauuguagu
ggaucuaauuuuuaagaaauuuugccuuuaaguuauuuuaccuguuuuuguuucuuguuuugaaagaugcgcauucuaaccuggaggucaauguuauguauu
uauuuauuuauuuauuugguucccuuccuauuccaagcuuccauagcugcugcccuaguuuucuuuccuccuuuccuccucugacuuggggaccuuuugggg
gagggcugcgacgcuugcucuguuuguggggugacgggacucaggcgggacagugcugcagcucccuggcuucuguggggccccucaccuacuuacccaggu
gggucccggcucugugggugauggggaggggcauugcugacuguguauauaggauaauuaugaaaagcaguucuggauggugugccuuccagauccucucug
gggcuguguuuugagcagcagguagccugcugguuuuaucugagugaaauacuguacaggggaauaaaagagaucuuauuuuuuuuuuuauacuuggcguuu
uuugaauaaaaaccuuuugucuuaaaaaaaaaaaaaaaaaaaaaaaaaaaaaaaaaaaaaaaaaaaa
AMINO ACID SEQUENCE OF THE 44 KD ISOFORM OF HUMAN PIM-1 KINASE

SEQUENCE ID NO.4

L P H E P H E P L T P P F S A L P D P A G A P S R R Q S R Q R P Q L S S D S P S A F R A S R S H S R N
A T R S H S H S H S P R H S L R H S P G S G S C G S S S G H R P C A D I L E V G M L L S K I N S L A H
L R A A P C N D L H A T K L A P G K E K E P L E S Q Y Q V G P L L G S G G F G S V Y S G I R V S D N L
P V A I K H V E K D R I S D W G E L P N G T R V P M E V V L L K K V S S G F S G V I R L L D W F E R P
D S F V L I L E R X E P V Q D L F D F I T E R G A L Q E E L A R S F F W Q V L E A V R H C H N C G V L
H R D I K D E N I L I D L N R G E L K L I D F G S G A L L K D T V Y T D F D G T R V Y S P P E W I R Y
H R Y H G R S A A V W S L G I L L Y D M V C G D I P F E H D E E I I R G Q V F F R Q R V S S E C Q H L
I R W C L A L R P S D R P T F E E I Q N H P W M Q D V L L P Q E T A E I H L H S L S P G P S K

### The 44 kD Isoform of Pim-1 Kinase (Pim-1L) is Expressed at High Levels in Human Prostate Cancer Tumors but not in Benign Prostate Hyperplasia

The presence of Pim-1L in human prostate cancer tissues was confirmed by immunohistochemical staining of a tissue array of prostate tumor and benign prostate hyperplasia. As shown in FIG. 2C, the level of Pim-1L is significantly higher in 11 out of 20 tumor tissue specimens in comparison to that in benign prostate hyperplasia (BPH) controls. In most of BPH tissues, very little Pim-1L was detected in luminal epithelial cells, though weak staining in the nucleus of basal cells was observed. By contrast, in the tumor tissues Pim-1L was detected in many luminal cells with intensive staining in the cytoplasm as well as the plasma membrane.

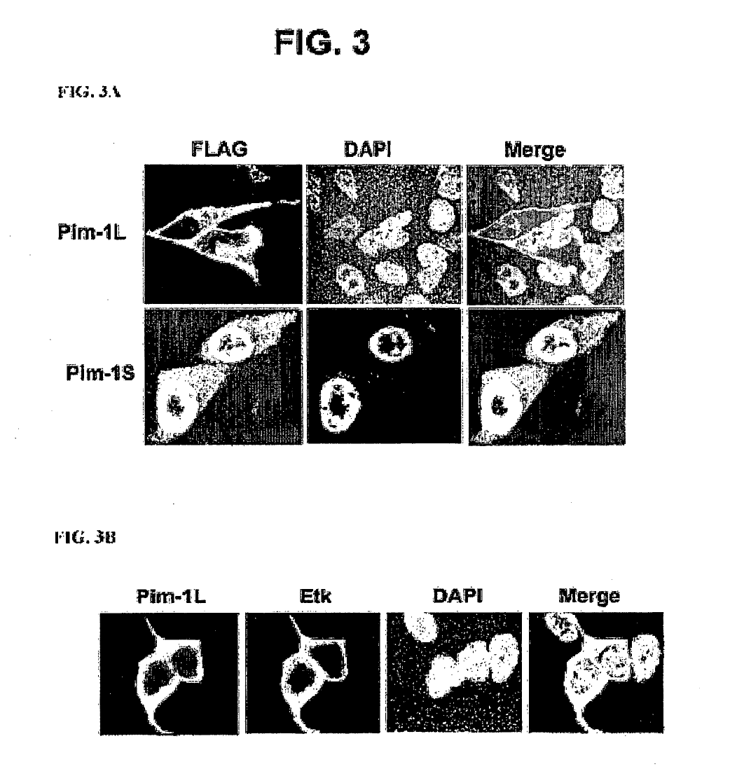

We also examined the localization of Flag-tagged Pim-1L and Pim-1S in the prostate cancer cell line LNCaP. As shown in FIG. 3A, Flag-tagged Pim-1L is primarily localized on the plasma membrane while Pim-1 S is predominantly present in the nucleus as previously reported (17, 20). Pim-1L is co-localized on the plasma membrane with Etk which is known to be recruited to the plasma membrane upon activation (21). Immunofluorescence staining was done with anti-Flag antibody and the confocal imaging was carried out as described in the Examples. The micrographs in FIG. 3B show the co-localization of Pim-1L and Etk in LNCaP cells transfected with Flag-tagged Pim-1L and T7-Etk. At 48 hour post-transfection, the cells were fixed and subjected to immunostaining. Pim-1L protein was detected by staining with anti-Flag antibody and Rhodamine labeled secondary antibodies. Etk protein was detected by staining with polyclonal anti-Etk antibody and FITC labeled secondary antibodies. The slides were analyzed by laser scanning confocal microscopy. The co-localization of the two proteins was detected by the merged of the confocal images. The results show that the 44 kD isoform (Pim-1L) is localized primarily on the plasma membrane while the 33 kD isoform is present in cytosol and nucleus, showing that these two isoforms may regulate distinct substrates in prostate cancer (PCA) cells.

### The PXXP Motif of 44 kD Isoform of Pim-1 Kinase Binds to the SH3 Domain of Etk in Prostate Cancer Cell Lines

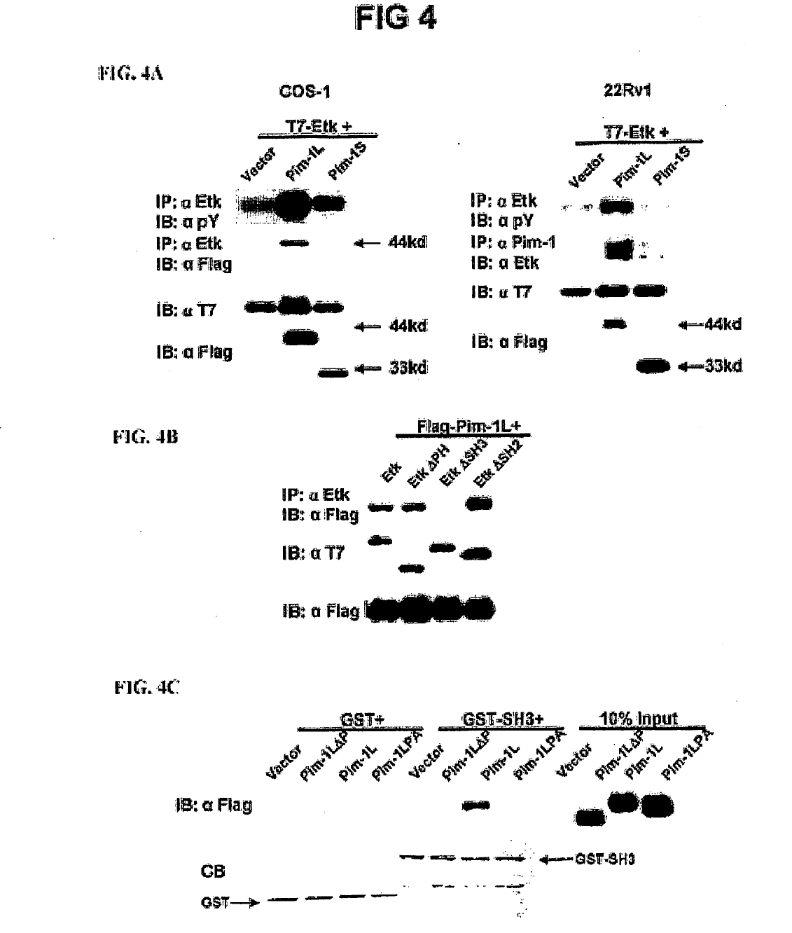

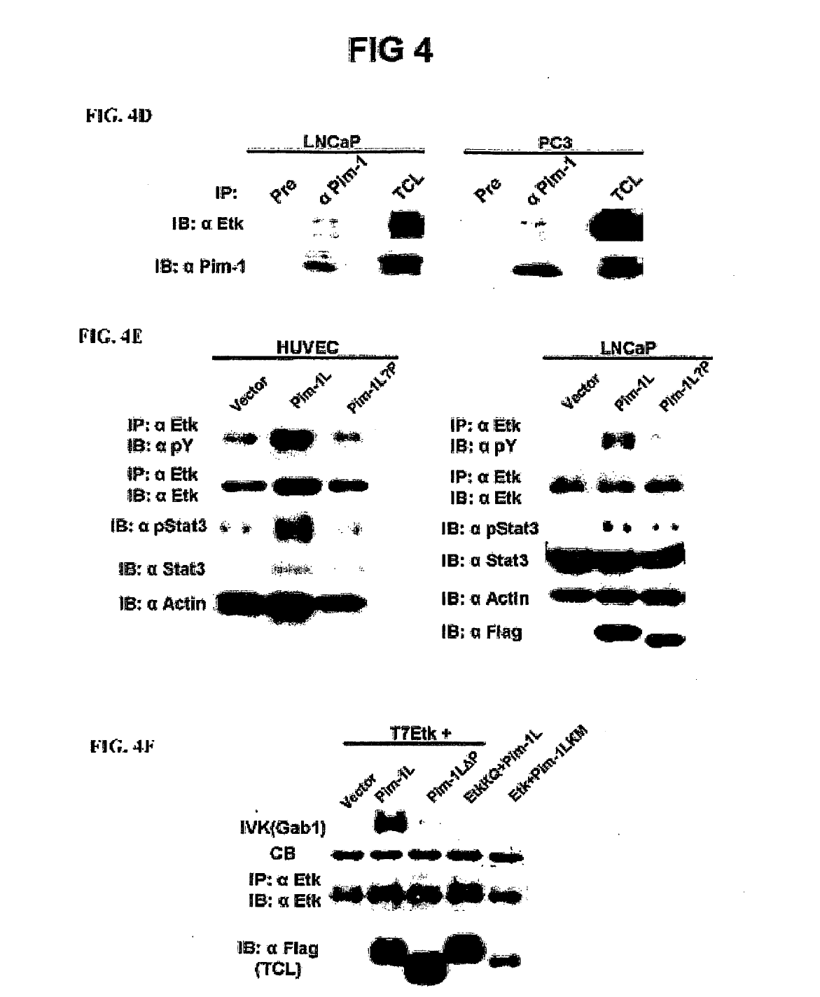

Both Etk and the 44 kD isoform (Pim-1L) have proline rich regions. In Etk the proline rich region is the SH3 domain and in Pim-1L it is the PXXP motif. Binding of Pim-1L to Etk was proved because Pim-1L co-immunoprecipitated with Etk bound to anti-Etk antibody in both COS-1 and 22Rv1 cells. By contrast, Pim-1S was barely detectable in the anti-Etk immunoprecipitates. FIG. 4A. The association of Pim-1L with Etk was accompanied by an unexpected robust increase of tyrosine phosphorylation of Etk. It is known that activation of Etk in HUVEC and LNCaP cells can induce tyrosine phosphorylation of Y705 in STAT3 (19).

To investigate whether the association of the 44 kD isoform and Etk is mediated by the interaction between the SH3 domain of Etk and the PXXP motif in the Pim-1L, a series of deletion mutants of Etk were co-transfected with the Flag-tagged Pim-1L. As shown in FIG. 4B, the deletion of the SH3 domain of Etk abolished the interaction of the two proteins. To test whether the association between Etk and Pim-1L is direct, GST-pull-down experiments (Glutathione S-Transferase) were carried out by incubating the immobilized GST-SH3 fusion protein with either 1) cell lysates expressing full-length Flag-tagged Pim-1L, 2) the mutant with the deletion (Pim-1LΔP) or 3) the mutation (Pim-1LPA) of both PXXP motifs. Only the Pim-1L associated with the GST-SH3 domain. Both mutants failed to bind (FIG. 4C), showing that the integrity of the PXXP motifs is required for the interaction between Pim-1L and Etk. The mutants containing deletion (Pim-1 LAP in which the first 15 amino acids are deleted) or mutation (Pim-1LPA in which Proline 2, 5, 8 and 11 are substituted by Alanines) of the PXXP motifs of human Pim-1L were generated by using the Quickchange Mutagenesis Kit (Stratagene).

The interaction between endogenous Etk and Pim-1L was also confirmed in two prostate cancer cell lines LNCaP and PC3 (FIG. 4D). The effects of Pim-1L on endogenous Etk activity in HUVEC and LNCaP cells were examined. FIG. 4E shows that ectopic expression (i.e, over-expression is driven by the CMV promoter) of Pim-1L in both cell lines resulted in elevated tyrosine phosphorylation of endogenous Etk and STAT3. Since it is known that activation of Etk in these cells can induce tyrosine phosphorylation of Y705 in STAT3, it is likely that Etk kinase activity is increased by Pim-1L but not by the mutant Pim-1LΔP. Activation of Etk kinase by the 44 kD isoform (Pim-1L) was confirmed by in vitro kinase assays of Etk as shown in FIG. 4F. Co-expression of Etk with Pim-1L enhanced the kinase activity of Etk as was evidenced by the increased tyrosine phosphorylation of the exogenous substrate Gab1. Deletion of the proline-rich PXXP motifs of Pim-1L (Pim-1LΔP) dramatically diminished its effects on the Etk kinase activity. Taken together, the data show that the integrity of both the SH3 domain of Etk and the proline-rich region of Pim-1L are required for the direct interaction of these two proteins and subsequent activation of Etk kinase activity by Pim-1L.

### The 44 kD Isoform (Pim-1L) Causes Drug Resistance in Prostate Cancer Cells By Binding to Etk Thereby Increasing Etk Activity

The results of the studies described herein demonstrate that Etk binds to the 44 kD isoform of Pim-1, through an interaction between the PXXP motif on the 44 kilodalton isoform (Pim-1L) and the SH3 domain of Etk. The binding of Pim-1L to the SH3 domain activates Etk kinase. Pim-1L thus competes with the tumor suppressor p53 for binding to Etk. The results further show that activation of Etk by binding to Pim-1L induces resistance to drug-induced apoptosis. Therefore, inhibiting the binding of Pim-1L to Etk has prophylactic and therapeutic uses in preventing and treating cancer by making the cancer more susceptible to apoptosis induced by chemotherapy, by permitting endogenous p53 to bind to Etk.

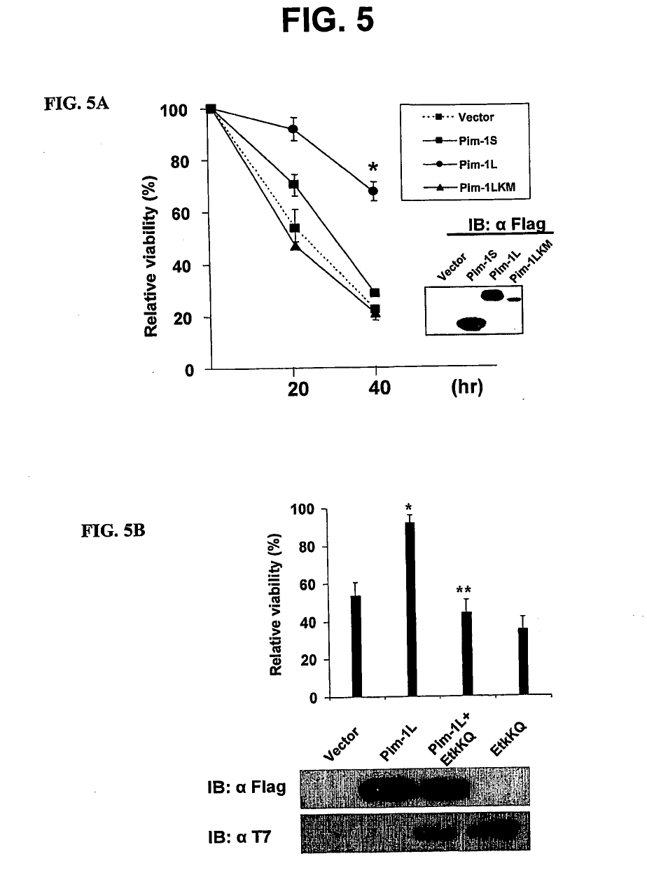

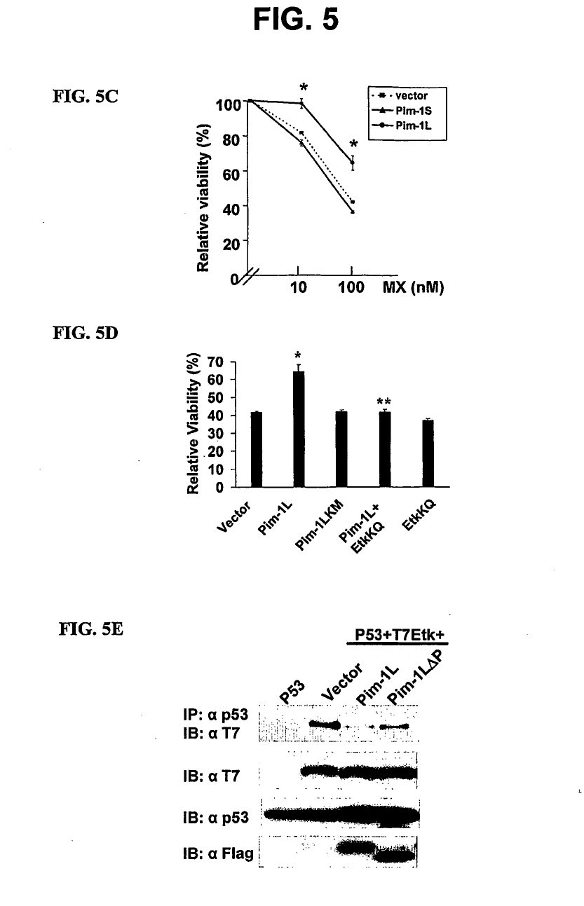

An increase of Etk activity confers drug resistance to apoptosis caused by chemotherapeutic agents in LNCaP cells (20), including doxorubicin and cisplatin. By contrast, P53 binding to Etk decreases Etk activity permits apoptosis. However, the mechanism by which Etk is activated was not known. The effects of Pim-1 on the drug response in LNCaP cells was studied by infecting the cells with lenti-viruses expressing Pim-1L (44 kD isoform), Pim-1S (33 kD isoform), Pim-1LKM (kinase inactive) or the empty vector control. As is shown in FIG. 5A, treatment of LNCaP cells with doxorubicin induced rapid apoptosis as was expected. By contrast, approximately 50% of the cells expressing the empty vector or the kinase-inactive Pim-1LKM remained viable at 20 hour post-treatment. However, the number of cells that survived in these two groups was drastically reduced to 20% after a 40 hour treatment. Over-expression of Pim-1S conferred some initial protection against drug-induced apoptosis with about 80% cell survival at 20 h. However, such protection was not sustained until 40 hours. In sharp contrast, LNCaP cells expressing the 44 kD Pim-1L isoform showed a remarkable and unexpectedly high drug resistance. Virtually no apoptotic cells were detected at 20 hours post-treatment in Pim-1L-expressing cells, and more than 70% of the cells expressing Pim-1L remained viable even after 40 hours of treatment. Thus it was discovered that the expression of the 44 kD Pim-1L isoform having three PXXP proline-rich regions significantly protects LNCaP cells from doxorubicin-induced apoptosis. the 33 kD Pim-1S isoform that does not have proline-rich regions affords only very limited protection. In this experiment, LNCaP cells were infected with Lenti-virus encoding Flag-Pim-1 S, Pim-1L, Pim-1LKM (kinase-deficient mutant) or vector control for 24h. The cells were then plated in 96-well plate and treated with doxorubicin (0.1 ug/ml) for the times as indicated in FIG. 5A.

To test whether the protection from chemotherapeutic agent-induced apoptosis by the 44 kD Pim-1L depends on Etk kinase activity, LNCaP cells were co-infected with Pim-1L and kinase-inactive EtkKQ simultaneously. FIG. 5B shows that kinase-inactive Etk significantly reduced the anti-apoptosis effects of Pim-1L, showing that tyrosine kinase activity of Etk is required for Pim-1L-induced drug resistance to apoptosis. Similar results were obtained when the effects of Pim-1 L on apoptosis induced by another chemotherapeutic drug Mitoxantrone were tested (FIGS. 5C & D).

It has been shown that the SH3 domain of Etk is capable of binding to the proline-rich domain of p53, and that the binding of p53 decreases Etk kinase activity more than 10 fold. Inhibition of Etk kinase activity in prostate cancer cells caused by the binding of p53 was shown to be essential for doxorubicin-induced p53-mediated apoptosis (20). It has now been discovered that the 44 kD Pim-1L isoform competes with p53 for binding to Etk, such that drug-resistance to apoptosis is conferred if the 44 kD isoform binds to and activates Etk. By contrast, susceptibility to drug-induced apoptosis is conferred if p53 binds to Etk inhibiting its activity. FIG. 5E shows that the interaction between Etk and p53 was disrupted by Pim-1L but not by Pim-1LΔP, showing a competition between the proline-rich domains of Pim-1L and p53 for binding to the SH3 domain of Etk. Taken together, our results show that the 44 kD Pim-1L isoform plays an important role in anti-apoptosis signaling in cancer cells in response to chemotherapeutic drugs by competing with p53 for binding to Etk.

The present invention is therefore directed to methods for treating or preventing cancer in an animal by inhibiting expression of Pim-1L or its ability to bind to and/or activate Etk. In one embodiment, this is accomplished by inhibiting the expression of 44 kD isoform in a cancer or precancerous cell by administering antisense or short interfering RNA nucleic acids that interfere with transcription or translation of Pim-1L. Such nucleic acids include isolated antisense nucleotides including DNA or RNA molecules that are sufficiently complementary to the gene (SEQ ID NO. 1, or biologically active fragment or variant thereof) or mRNA for Pim-1L (SEQ ID NO. 2, or biologically active fragment or variant thereof) to permit specific hybridization to the gene or mRNA under physiologic conditions thereby inhibiting expression of Pim-1L by inhibiting transcription or translation, respectively. Sufficiently complementary means that there is enough complementarity to the gene (or mRNA) to permit specific hybridization under physiologic conditions thereby interfering with Pim-1L expression.

In some embodiments the antisense DNA or RNA includes a sequence complementary to the entire gene or several hundred nucleotides thereof, however antisense is often from about 8 to about 50 nucleotides long, or 8 to 100 nucleotides in length. There is no arbitrary limit on length other than the ability of the antisense to specifically hybridize to the target DNA or RNA molecule thus interfering with the normal function of the target DNA or RNA to cause a loss of utility. There should be a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed. The stringency of hybridization of antisense is discussed below and varies according to the use (in vivo or in vitro or in assays). The nucleic acid and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the nucleic acid and the DNA or RNA target. A more thorough discussion of complementarity and hybridization is presented below.

Identification of antisense nucleotides (RNA or DNA) is straightforward since the gene sequence encoding the Pim-1 kinase in humans is known. The gene sequence encoding Pim-1 kinase is identified as SEQ. ID NO. 1; mRNA is SEQ ID NO. 2. The amino acid sequence for human Pim-1 kinase is described herein as SEQ. ID NO. 4. Antisense technology and short interfering RNAs are well known in the art and are described in detail below.

The antisense compounds of the present invention, either alone or in combination with other antisense compounds or therapeutics, can be used as tools in differential and/or combinatorial analyses to elucidate expression patterns of Pim-1L expressed within cells and tissues.

In another embodiment of the invention, the gene or messenger RNA for the Pim-1L isoform (or variant or biologically active fragment thereof) is used in a screening assay to identify compounds that bind to the gene or to messenger RNA in a way that interferes with their expression. In another embodiment, the human 44 kD protein isomer itself is used in a drug screen to identify compounds that bind to and interfere with the ability of the 44 kD isomer to bind to activate Etk.

Most of the studies on Pim-1 have been focused on the 33 kD isoform. The results presented herein are the first to demonstrate that the 44 kD isoform of Pim-1 is expressed in all human prostate cancer cell lines tested and is significantly upregulated in human prostate tumor specimens in comparison to the benign prostatic hyperplasia controls. Although the alternative translation initiation site CUG in human Pim-1 gene is not present in an optimal Kozak consensus context as is its mouse counterpart, the 44 kD (Pim-1L) isoform is encoded efficiently and is detectable in all human prostate cancer cell lines tested as well as in human prostate tumor specimens.

The human Pim-1 gene is mapped to the fragile chromosomal site 6p21 where aberrant translocations were reported in acute nonlymphocytic leukemia (2). The levels of Pim-1 mRNA are often elevated in human leukemias (3). Further, transgenic mice over-expressing the Pim-1 gene are tumor-prone and susceptible to carcinogens and other tumor promoters, showing a direct role of Pim-1 kinase in oncogenesis (4-5). For the forgoing reasons and because p53 is found in all forms of cancer, the present inventions related to inhibiting expression of the 44 kD isoform or interfering with its ability to bind to and/or activate Etk are widely applicable to treating or preventing other forms of cancer. Thus the results of the in vitro experiments presented here support the role of the 44 kD isoform in conferring drug resistance to apoptosis in human cancer cells.

It is noteworthy that Pim-1L is expressed at the highest level in PC3 cells. The relative ratio between the level of Pim-1L to Pim-1 S is also highest in PC3 cells for both the endogenous Pim-1 and the ectopically expressed EST clones. At present, it is not clear whether the relatively high level of Pim-1L in PC3 cells is due to the increased stability of Pim-1L or the enhanced efficiency of usage of the alternative CUG translation initiation site. The PC3 cell line is known for its remarkable resistance to various apoptosis inducers.

The Pim-1L contains three PXXP motifs in its unique N-terminal sequence, which can compete with the proline-rich motifs of p53 for binding to the SH3 domain of tyrosine kinase Etk. Our previous study showed that down-regulation of Etk activity by p53 is necessary for drug-induced DNA damage and apoptosis in LNCaP cells. Therefore, disruption of the p53 binding to Etk by the 44 kD isoform (Pim-1L) and the Pim-1L-induced activation of Etk tyrosine kinase activity confers resistance to chemotherapeutic drugs in prostate and other cancer cells. Because the 44 kD Pim-1L isoform is primarily localized on the plasma membrane as opposed to an intracellular compartment it will be accessible to drugs that can inhibit its ability to bind to or activate Etk.

Certain embodiments are directed to an expression vector carrying a gene encoding an antisense nucleic acid that is sufficiently complementary to DNA encoding the 44 kilodalton isoform or a biologically active fragment or variant thereof to permit specific hybridization under physiologic conditions thereby inhibiting or inhibiting Pim-1L expression, for example in an animal cancer cell or cancer cell line. Such vectors can be used to transform cells thereby increasing or restoring drug-induced apoptosis to study the biological activity of Pim-1L and to assay for compounds that interfere with its expression or activity. Certain other embodiments are directed to a host cell or organism transformed or transfected with this expression vector. Certain embodiments of the invention include incorporation of the DNA molecule (such as antisense DNA or RNA) into an expression vector, an autonomously replicating plasmid, a virus (e.g., a retrovirus, lenti-virus, adenovirus, or herpes virus), or into the genomic DNA of a prokaryote or eukaryote.

In addition to binding to and activating Etk kinase, localization of the Pim-1L on the plasma membrane may also allow it to phosphorylate other membrane or membrane-associated proteins which are directly involved in drug resistance. This is discussed below in the context of ABC transporters.

### Phosphorylation of Breast Cancer Resistance Protein (BCRP) by the 44 kD Isoform (Pim-1L) Confers Resistance to Chemotherapeutic Drugs

BCRP is a xenobiotic transporter which is over-expressed in a variety of drug-resistant human cancer cell lines, and confers resistance to many chemotherapeutic agents. BCRP is an about 655 amino acid protein and is encoded by a gene which has about 2418 nucleotides. The protein demonstrates activity and has a sequence homology which places it in the ATP-binding cassette (ABC) superfamily of transporter proteins (hereafter “the ABC transporters”). The molecular mass is approximately 72.3 kilodaltons (kD) exclusive of any glycoylation. Expression of BCRP in drug-sensitive human cancer cells confers resistance to mitoxantrone, doxorubicin, and daunorubicin, and reduces daunorubicin accumulation in the cloned transfected cells. The BCRP was shown to be over-expressed in human multi-drug resistant (MDR) breast carcinoma cells MCF-7, colon carcinoma cells S1, HT29, gastric carcinoma cells EPG85-257, fibrosarcoma cells EPR86-079, and myeloma 8226 cells. U.S. Pat. No. 6,313,277, the entire contents of which are hereby incorporated by reference as if fully set forth herein.

To search the potential protein targets for Pim-1L kinase, yeast two-hybrid screening was conducted using the kinase-inactive Pim-1L (Pim-1LKM) as bait. It was discovered that Pim-1L kinase binds to human BCRP (Breast Cancer Resistance Protein) synthesized from cDNA obtained from the HeLa cDNA library. The interaction of Pim-1L and BCRP was not previously known. BCRP, also known as ABCG2, has been described in WO 99/40110, US published application 2003/036645 and U.S. Pat. No. 6,313,277, the entire contents of which are hereby incorporated by reference as if fully set forth herein. BCRP, first discovered as an amplified gene in breast cancer cells resistant to doxorubicin, is an ATPase transporter that transports a variety of chemotherapeutic drugs out of cells thereby conferring on cancer cells chemotherapy drug resistance. BCRP is expressed in many tissues and over-expression in many cell types results in resistance to chemotherapeutic drug-induced apoptosis (23).

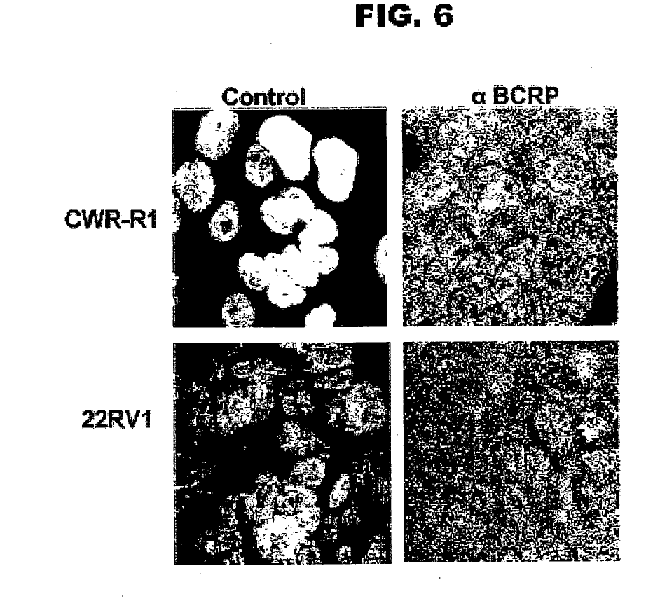

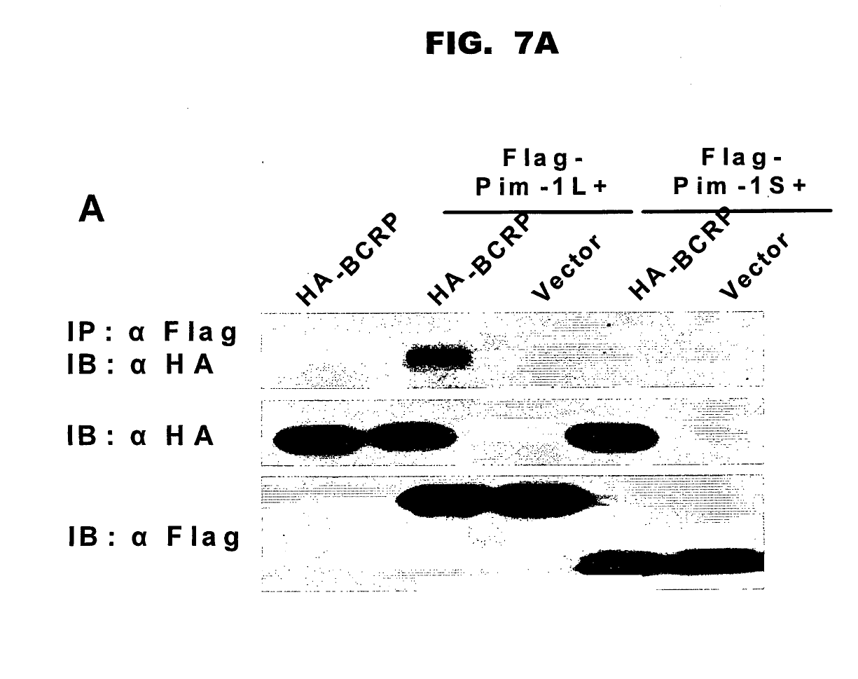

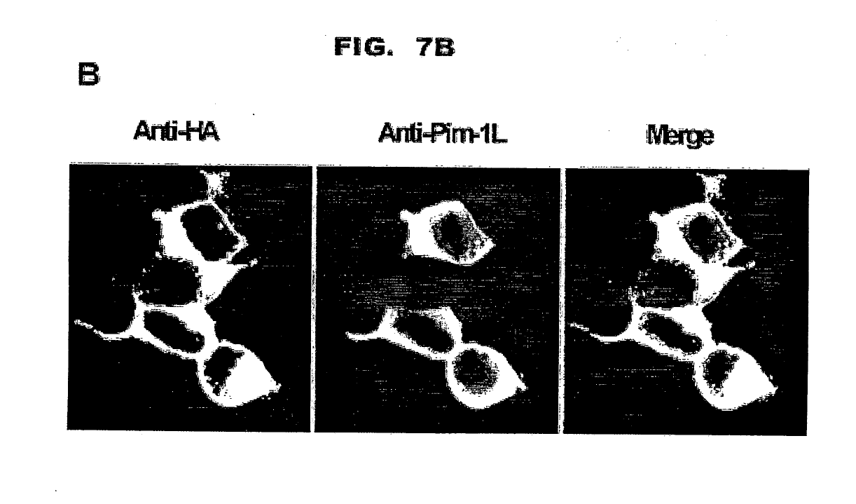

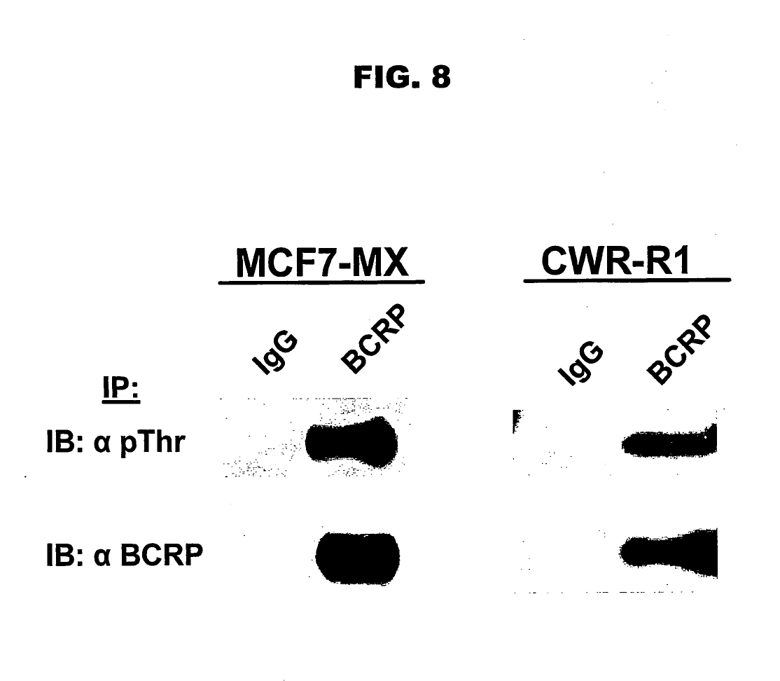

To determine whether BCRP is expressed in human prostate cancer cell lines, both CWR-R1 and 22Rv1 cells were immunostained with the anti-BCRP antibody. As shown in FIG. 6, BCRP is expressed in both CWR-R1 and 22Rv1 cells. The interaction between these two proteins is further supported by the co-localization of ectopically expressed FLAG-tagged Pim-1L and HA-tagged BCRP in LNCaP cells (FIG. 7). (HA=Human influenza haemagglutinin protein). The interaction between Pim-1L and BCRP in CWR-R1 cells was confirmed by the co-immunoprecipitation experiments shown in 293T cells (FIG. 7A), and by immunostaining showing their co-localization on the plasma membrane in LNCaP cells (FIG. 7B). In addition, endogenous BCRP was shown to be phosphorylated at threonine residue(s) in both breast (MCF7/MX) and prostate (CWR-R1) cancer cell lines (FIG. 8). Furthermore, threonine phosphorylation of endogenous BCRP increased when the kinase active Pim-1L (but not the kinase-inactive Pim-1LKM) was over-expressed in MCF7/MX cells (FIG. 9), showing that Pim-1L can induce BCRP phosphorylation.

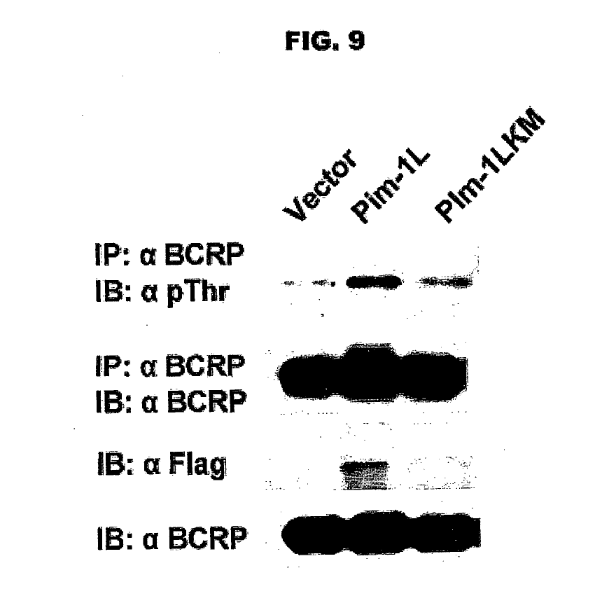

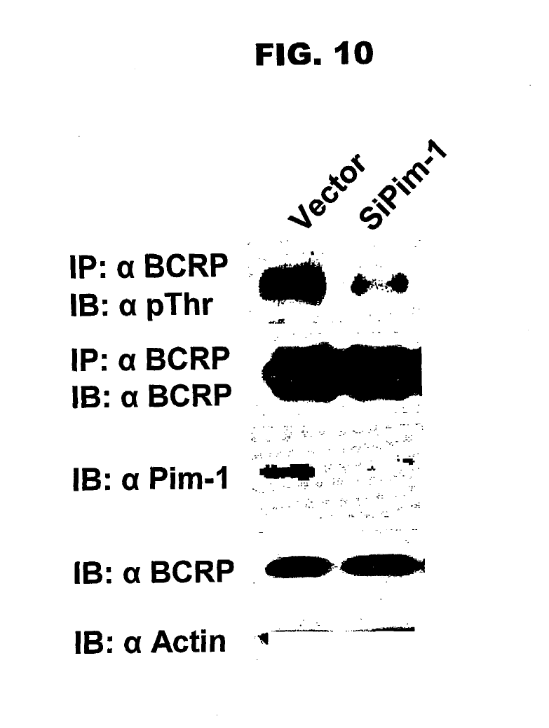

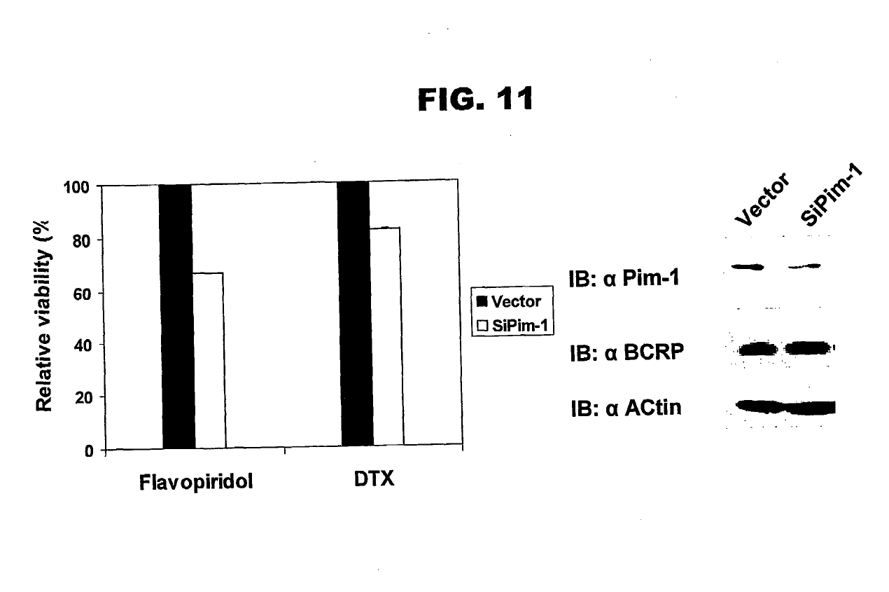

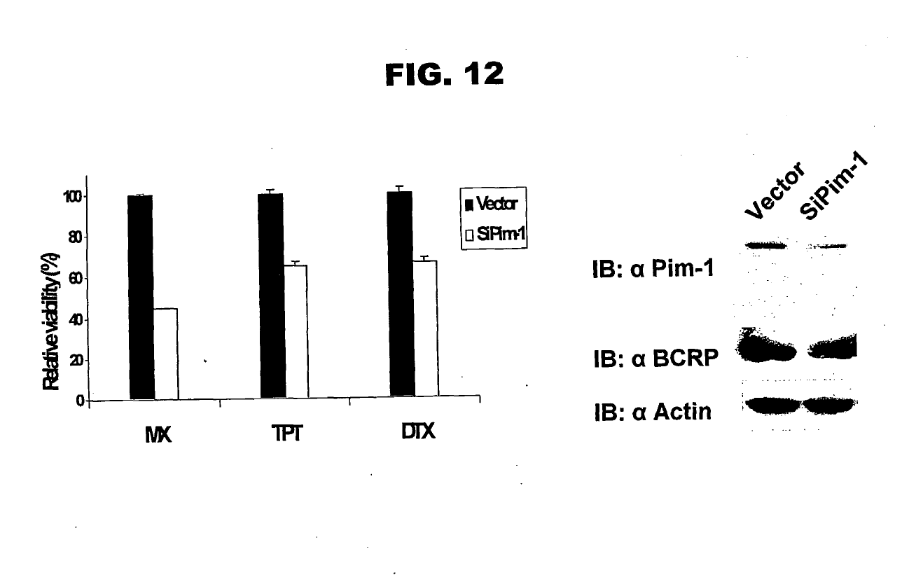

The phosphorylation of BCRP is therapeutically significant because it prevents BCRP from causing resistance to chemotherapeutic agents. This was corroborated by our observation that inhibiting Pim-1L in MCF7/MX cells with a specific short interfering RNA (siRNA) that targets Pim-1L significantly reduced the phosphorylation of BCRP (FIG. 10) and inhibited BCRP-mediated drug resistance (FIG. 11). Downregulating Pim-1L expression by a specific siRNA also sensitized the prostate cancer cell line CWR-R1 to apoptosis induced by the chemotherapeutic agents mitoxantrone, topotecan and docetaxel (FIG. 12). The siRNA target sequence for inhibiting Pim-1L expression used to inhibit Pim-1L expression is sequence is: 5′ GCAGGACAGUGCUUGAUAC 3′ identified as SEQ ID NO. 3, as is described in a previous study (29, the entire contents of which is hereby incorporated by reference as if fully set forth herein). This siRNA is targeted at nucleotides 1540-1558 of SEQ ID NO. 1.

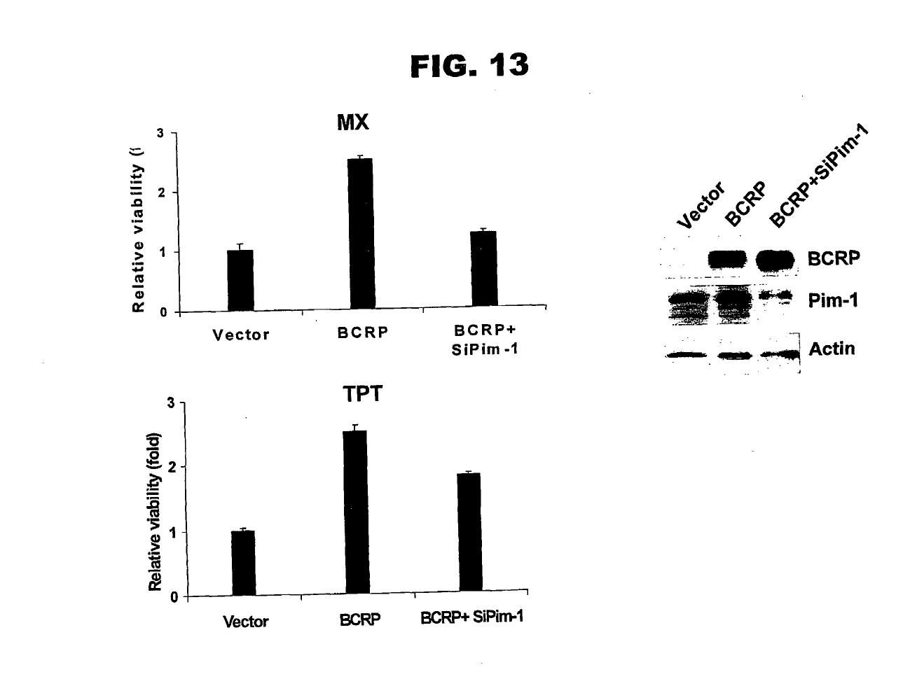

This evidence shows that short interfering RNA (and by analogy also antisense RNA or DNA) can be used therapeutically to inhibit BCRP-mediated drug resistance. Additional experiments showed that over-expression of BCRP in LNCaP prostate cancer cells increased resistance to mitoxantrone and topotecan. Importantly, inhibition of Pim-1L by a specific siRNA (SEQ ID.NO. 3) was able to reduce or compromise BCRP-mediated resistance to these drugs even in cells that over-express BCRP (FIG. 13).

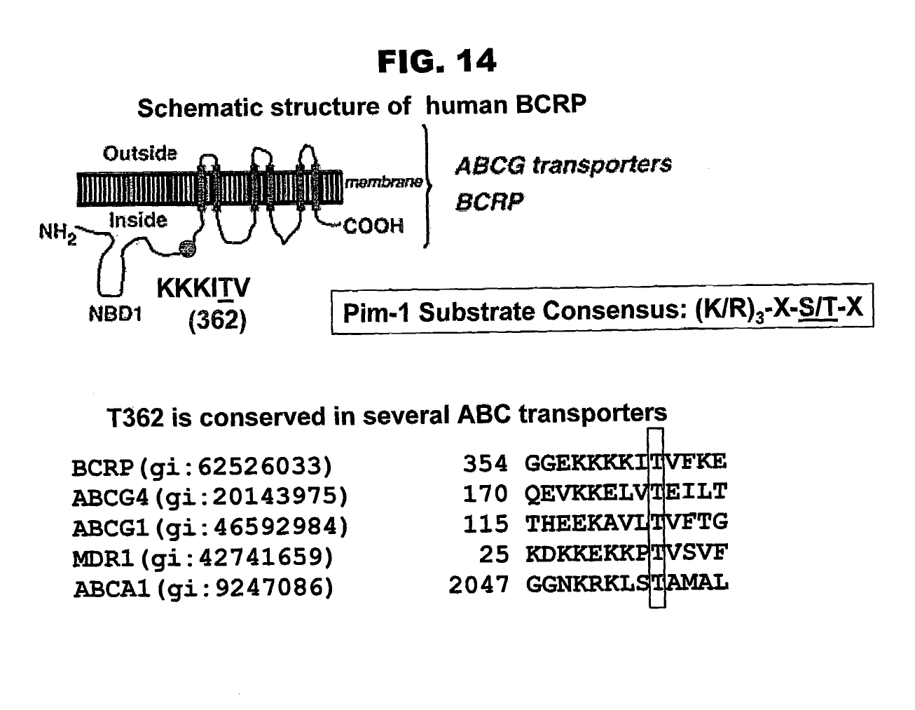

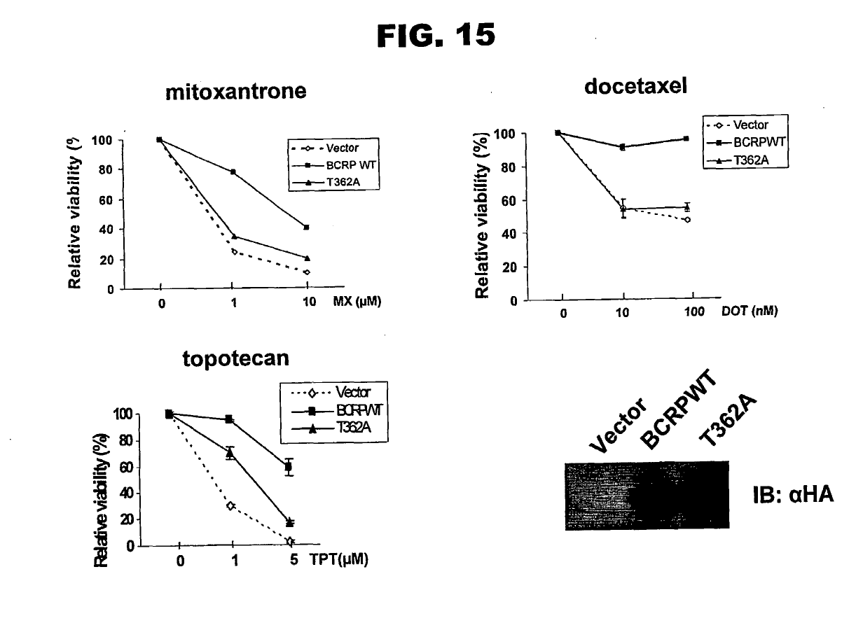

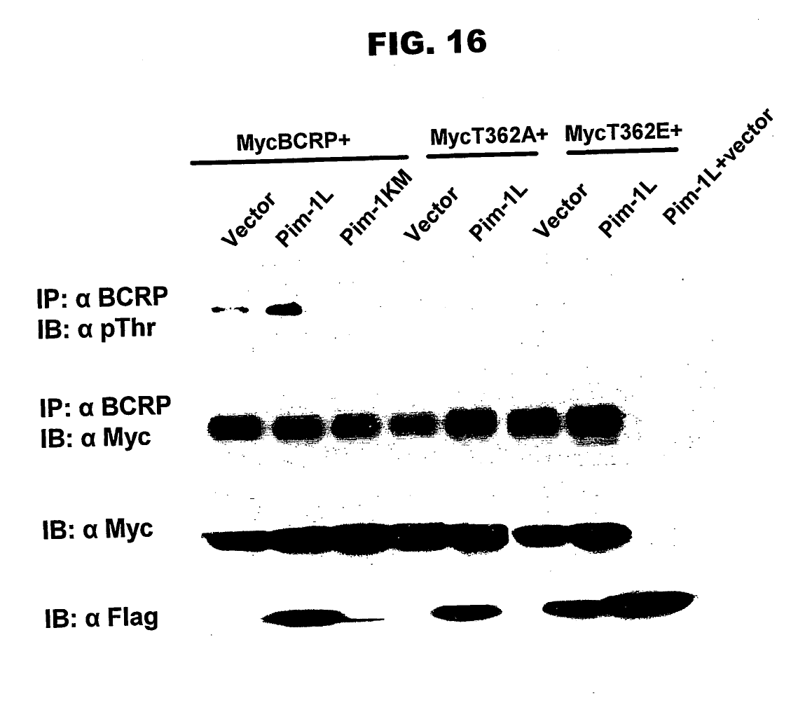

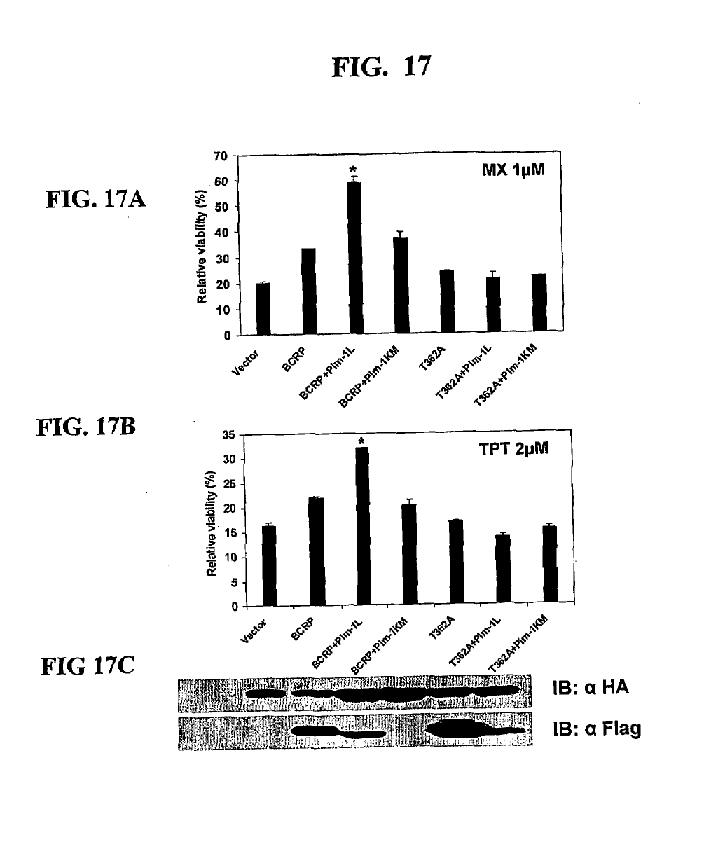

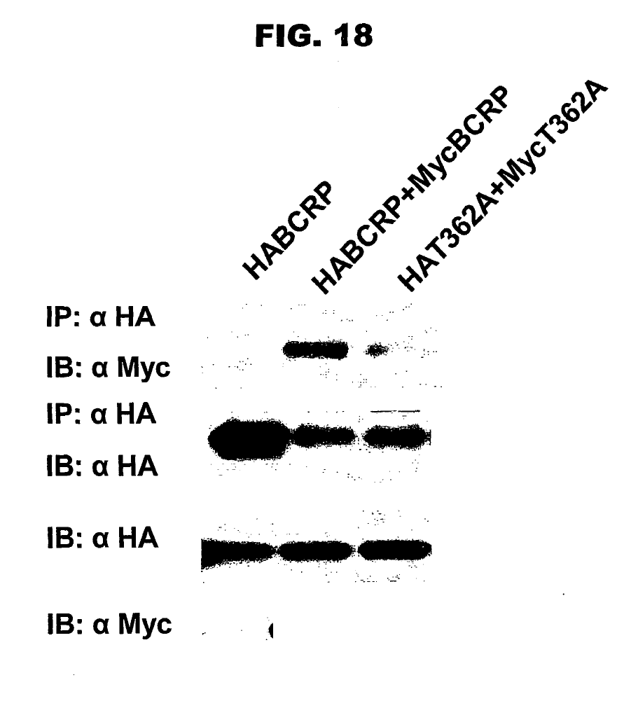

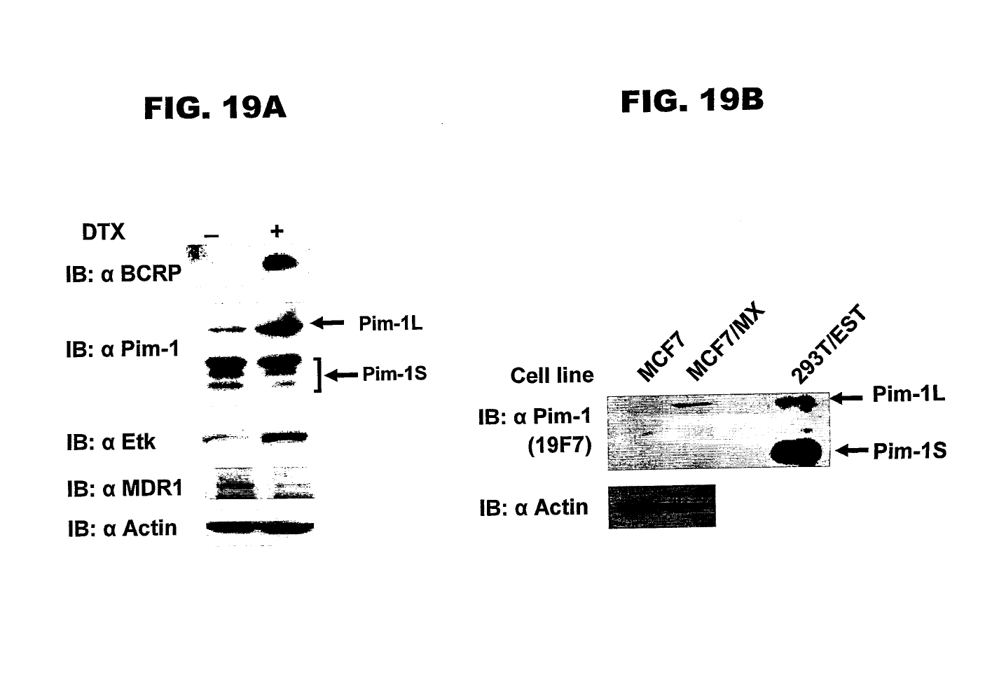

BCRP is known to contain an amino acid sequence that matches the consensus sequence of the preferred substrates of Pim-1: (K/R)₃XS/TX (where X stands for any residue and T=threonine) (FIG. 14A) (24). To test whether phosphorylation of threonine in BCRP is required for BCRP-mediated drug resistance, we substituted residue number 362 which is Threonine (hereafter “T362”) of BCRP with Ala. We discovered that this T362 substitution compromised BCRP-mediated drug resistance in LNCaP cells to all drugs tested (FIG. 15), showing the integrity of the T362 residue of BCRP is important for its activity. We conducted more experiments to determine whether Pim-1L causes BCRP phosphorylation at the T362 residue. 293T cells (obtained from the ATCC) were co-transfected with HA-tagged BCRP or a mutant form: T362A (alanine) or T362E (glutamate), and with Pim-1L or the kinase-inactive Pim-1LKM form. As shown in FIG. 16, co-expression of BCRP with Pim-1L, but not the kinase-inactive mutant Pim-1LKM, resulted in increased threonine phosphorylation of BCRP. Substitution of T362 with either alanine or glutamate completely abolished threonine phosphorylation of BCRP, showing that T362 is the site of phosphorylation regulation. We noted that there was a basal level of threonine phosphorylation of BCRP in the vector-transfected 293T cells, which may be caused by a low level of endogenous Pim-1 activity; such phosphorylation was inhibited by over-expression of the kinase-dead Pim-1LKM. It is possible that the mutant Pim-1LKM competes with endogenous active Pim-1L for binding to and phosphorylation of BCRP. Importantly, we also discovered that a threonine residue is embedded in the consensus sequence (K/R)₃XS/TX in several other ABC transporters ABCG4, ABCG1, MDR1 or ABCA1 as is shown in FIG. 14B. This means that Pim-1L inhibition can prevent phosphorylation of other ABC transporters thereby suppressing drug resistance.

To summarize, it has been shown that BCRP is expressed in prostate cancer cells, and that phosphorylation of the T362 residue of BCRP by Pim-1L is responsible for causing drug-induced resistance to apoptosis. Therefore inhibiting expression of Pim-1L prevents drug resistance in cancer cells in two ways: 1—by preventing Pim-1L from binding to and activating Etk, and 2—by preventing phosphorylation of BCRP or other ABC transporters. Certain embodiments of the invention are directed to treating or preventing cancer by inhibiting Pim-1L-induced phosphorylation of BCRP or other ABC transporter, including inhibiting phosphorylation of the T362 residue on BCRP using antisense technology described above.

Another embodiment of the invention is directed to methods for treating or preventing cancer by inhibiting expression of BCRP or other ABC transporter in cancer cells, for example by using nucleic acids (including antisense DNA and RNA and short interfering RNA) that are sufficiently complementary to hybridize under physiologic conditions to the gene or messenger RNA encoding BCRP or other ABC transporters thereby inhibiting their expression. Phosphopeptides, for example those corresponding to the region containing T362 of BCRP, interfere dimerization of BCRP, thereby preventing BCRP-induced drug resistance. Certain embodiments are directed to phosphopeptides that inhibit phosphorylation or dimerization of BCRP and the other ABC transporters, and to methods of treating or preventing cancer with such phosphopeptides.

### Drug Screening Assays

According to certain embodiments of the invention, the gene encoding the 44 kD isomer of Pim-1 kinase or a biologically active fragment of the gene, is used as a marker to screen for compounds that inhibit expression of the gene, messenger RNA or gene product. In another embodiment messenger RNA encoding Pim-1L (or a biologically active fragment or variant thereof) is used in a screening assay to identify compounds that inhibit expression of the messenger RNA or of Pim-1L. In another embodiment a fragment of the gene encoding one or more PXXP proline-rich regions of Pim-1L is used to detect compounds that inhibit the biological activity of Pim-1L, i.e. its ability to activate Etk. In an embodiment detection of such compounds is facilitated by coupling the gene or gene portion, or a homologue thereof, to a suitable reporter, e.g. a fluorescent reporter molecule. Suitable screening systems include, but are not limited to Northern blots, RT-PCR using specific primers and probes for the gene, solution hybridization and RNAase protection assays. Large scale screening, e.g., so called high throughput screening (HTS) of chemical and/or biologic libraries can be performed with a reporter system as described above. In another embodiment the assay for compounds that regulate gene expression is constructed as an assay suitable for high throughput (HTP) screening, for example an assay adapted for the commonly used 96-well format, the 384-well format or denser formats, such as micro arrays or chips, carrying immobilized reagents on their surface.

In another embodiment, the gene or mRNA for the 44 kD isoform of Pim-1 or a fragment of the gene is used to screen for compounds that inhibit the ability of the 44 kD isoform to phosphorylate breast cancer resistance protein or other ABC transporters. In a specific embodiment, the gene for the 44 kD isoform is used to screen for compounds that inhibit the ability of the 44 kD isoform to phosphorylate T362 of BCRP.

### DEFINITIONS

Isolated Pim-1 kinase nucleic acid molecules are at least 10 to over 1,000 nucleotides in length (e.g., 10, 20, 50, 100, 200, 300, 400, 500, 1000, or more nucleotides in length). In some embodiments, isolated Pim-1 kinase nucleic acid molecules are between 150 and 370 nucleotides in length (e.g., 150, 175, 200, 225, 250, 275, 300, 325, 350, or 370 nucleotides in length). As described below, the full-length human Pim-1 kinase gene transcript contains 6 exons and is 5000 nucleotides in length, with a coding region that is 1215 nucleotides in length SEQ ID NO. 1 (also GeneBank Identification No. 18044377). The full gene transcript includes introns and other untranslated regions of the gene. The full-length mouse transcript is 2032 nucleotides in length (GeneBank Identification No. 40254619), with a coding region that is 941 nucleotides in length (nucleotides 333 to 1274 of GeneBank Identification No. 40254619). A human Pim-1 kinase nucleic acid molecule therefore is not required to contain all of the coding region listed in SEQ ID NOS: 1, or all of the exons. In fact, an Pim-1 kinase nucleic acid molecule can contain as little as a single exon or a portion of a single exon (e.g., 10 nucleotides from a single exon). Nucleic acid molecules that are less than full-length can be useful, for example, for diagnostic purposes or as antisense nucleic acids.

### Antisense Nucleic Acids

Antisense-RNA and anti-sense DNA have been used therapeutically in mammals to treat various diseases. See for example Agrawal, S, and Zhao, Q. (1998) Curr. Opin. Chemical Biol. Vol. 2, 519-528; Agrawal, S. and Zhang, R. (1997) CIBA Found. Symp. Vol. 209, 60-78; and Zhao, Q, et al., (1998), Antisense Nucleic Acid Drug Dev. Vol 8, 451-458; the entire contents of which are hereby incorporated by reference as if fully set forth herein. Antisense oligodeoxyribonucleotides (antisense-DNA) and oligoribonucleotides (antisense-RNA) can base pair with a gene, or its mRNA transcript. An antisense PS-oligodeoxyribonucleotide for treatment of cytomegalovirus retinitis in AIDS patients is the first antisense oligodeoxyribonucleotide approved for human use in the US. Anderson, K. O., et al., (1996) Antimicrobiol. Agents Chemother. Vol. 40, 2004-2011, and U.S. Pat. No. 6,828,151 by Borchers, et al. entitled Antisense modulation of hematopoietic cell protein tyrosine kinase expression describes methods for making and using antisense-nucleic acids and their formulation; the entire contents of which are hereby incorporated by reference as if fully set forth herein.

Others have shown that antisense nucleic acids complementary to the gene for glutamine synthetase mRNA in Mtb effectively enter the bacteria, complex with the mRNA and inhibit glutamine synthetase expression, the amount of the poly-L-glutamate/glutamine component in the cell wall, and bacterial replication in vitro. Harth, G., et al., PNAS Jan. 4, 2000, Vol. 97, No. 1, P 418-423, the entire contents of which are hereby incorporated by reference as if fully set forth herein.

The present invention is directed in part to inhibiting expression or activity of Pim-1L by inhibiting transcription, for example, using antisense technology. However, as would be appreciated by the skilled practitioner, any other suitable method may be utilized. Other methods of inhibiting Pim-1L activity may utilize antibodies or binding polypeptides (such as phosphopeptides discussed herein that bind to proline rich regions of Pim-1K) or other small molecules which, for example, bind or inhibit the binding region of Pim-1K or ABC transporters. As used herein, the term “antisense nucleotide” or “antisense” describes a nucleic acid including an oligoribonucleotide, oligodeoxyribonucleotide, modified oligoribonucleotide, or modified oligodeoxyribonucleotide which specifically hybridizes under physiological conditions to DNA encoding the polypeptide of the invention or to an mRNA transcript of the gene and, thereby, inhibits the transcription of that gene and/or translation of mRNA. Antisense technology can be used to control gene expression through triple-helix formation of antisense DNA or RNA, both of which methods are based on binding of a polynucleotide to DNA or RNA. For example, the 5′ coding portion or the mature protein sequence, which encodes for the protein of the present invention, is used to design an antisense RNA nucleic acid of from 10 to 500 base pairs in length, preferably from 10-100, most preferably from 10-50. The only limit on the size of the antisense is its ability to hybridize with the gene or mRNA and inhibit expression of Pim-1L or ABC transporters. The antisense RNA nucleic acid specifically hybridizes to the mRNA in vivo and inhibits translation of an mRNA molecule into the protein (antisense—Okano, J. Neurochem., 56:560 (1991); Oligodeoxynucleotides as Antisense Inhibitors of Gene Expression, CRC Press, Boca Raton, Fla. (1988)). A DNA nucleic acid is designed to be complementary to a region of the gene involved in transcription (triple-helix—see Lee et al. Nucl. Acids Res., 6:3073 (1979); Cooney et al., Science, 241:456 (1988); and Dervan et al., Science, 251: 1360 (1991), thereby preventing transcription and the production of the polypeptide.

Methods of making antisense-nucleic acids are well known in the art. Further provided are methods of modulating the expression of the 44 kD isoform and associated gene and mRNA in cells or tissues by contacting the cells or tissues with one or more of the antisense compounds or compositions of the invention. As used herein, the terms “target nucleic acid” encompass DNA encoding the 44 kD isoform, RNA (including pre-mRNA and mRNA) transcribed from such DNA, and also cDNA derived from such RNA. The specific hybridization of an oligomeric compound with its target nucleic acid interferes with the normal function of the target nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, and catalytic activity which may be engaged in or facilitated by the RNA, Etk and ABC transporter phosphorylation. The overall effect of such interference with target nucleic acid function is modulation of the expression of the 44 kD isoform. In the context of the present invention, “modulation” means (inhibition) in the expression of the 44 kD isoform gene. In the context of the present invention, inhibition is the preferred form of modulation of gene expression and the gene and mRNA are preferred targets.

The antisense nucleic acids of the present invention are specifically targeted to the 44 kD isoform gene and the mRNA for it. The sequence of the sense or coding strand of the gene for the 44 kD isoform is shown as SEQ ID NO. 1. The targeting process includes determination of a site or sites within the target gene for the antisense interaction to occur such that the desired inhibitory effect. Within the context of the present invention, a preferred intragenic site is the region encompassing the translation initiation or termination codon of the open reading frame (ORF) of the gene. The translation initiation codon is typically 5′-AUG (in transcribed mRNA molecules; 5′-ATG in the corresponding DNA molecule). However the 44 kD isoforms of hPim-1 has an alternative translational initiation codon CTG (Large font, bold, all caps in SEQ ID NO. 1). Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene, particularly the human 44 kD isoform.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively with antisense nucleic acids or short-interfering RNA. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The untranslated region of the 44 kD isoforms is the first 227 nucleotides of SEQ ID NO. 1.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

Once one or more target sites have been identified, nucleic acids are chosen that are sufficiently complementary to the target, i.e., specifically hybridizes to give the desired effect of inhibiting gene expression and transcription under physiologic conditions where the nucleic acids are used therapeutically or prophylactically.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the nucleic acid and the DNA or RNA target.

It is understood in the art that the sequence of an antisense compound need not and is often not 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense and other compounds of the invention that specifically hybridize to the target and inhibit expression of the target are identified through routine experimentation, and the sequences of these compounds are herein below identified as preferred embodiments of the invention. The target sites to which these preferred sequences are complementary are herein below referred to as “active sites” and are therefore preferred sites for targeting. The PXXP motif of the 44 kD isoforms is an active site, for example. Therefore another embodiment of the invention encompasses compounds which specifically hybridize to these active sites.

Antisense compounds are commonly used as research reagents and diagnostics. For example, antisense nucleic acids, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense nucleic acids have been employed as therapeutic moieties in the treatment of disease states in animals and man. Antisense nucleic acid drugs, including ribozymes, have been safely and effectively administered to humans and numerous clinical trials are presently underway. It is thus established that nucleic acids can be useful therapeutic modalities that can be configured to be useful in treatment regimes for treatment of cells, tissues and animals, especially humans.

While antisense nucleic acids are a preferred form of antisense compound, the present invention comprehends other oligomeric antisense compounds, including but not limited to nucleic acid mimetics. The antisense compounds in accordance with this invention preferably comprise from about 8 to about 50 nucleobases (i.e. from about 8 to about 50 linked nucleosides). Particularly preferred antisense compounds are antisense nucleic acids, even more preferably those comprising from about 12 to about 30 nucleobases. Antisense compounds include ribozymes, external guide sequence (EGS) nucleic acids (oligozymes), and other short catalytic RNAs or catalytic nucleic acids which specifically hybridize to the target nucleic acid and modulate its expression.

The antisense compounds used in accordance with this invention may be conveniently and routinely made through the well-known technique of solid phase synthesis. Equipment for such synthesis is sold by several vendors including, for example, Applied Biosystems. (Foster City, Calif.). Any other means for such synthesis known in the art may additionally or alternatively be employed. It is well known to use similar techniques to prepare nucleic acids such as the phosphorothioates and alkylated derivatives.

The antisense compounds of the invention are synthesized in vitro and do not include antisense compositions of biological origin. Certain embodiments do cover genetic vector constructs designed to direct the in vivo synthesis of antisense molecules in specifically targeted cells like cancer cells. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the present invention can be utilized for diagnostics, therapeutics, prophylaxis and as research reagents and kits. For therapeutics, an animal, preferably a human, suspected of having a disease or disorder (especially cancer) that can be treated by modulating the expression of the 44 kD isoform is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

An antisense molecule capable of hybridizing to the nucleic acid under physiologic conditions according to the invention may be used as a probe or as a medicament or may be included in a pharmaceutical composition with a pharmaceutically acceptable carrier, diluent or excipient therefore to treat the particular lymphoma. Nucleic acid molecules according to the invention may be inserted into the vectors described in an antisense orientation in order to provide for the production of antisense RNA. Antisense RNA or other antisense nucleic acids, including antisense peptide nucleic acid (PNA), may be produced by synthetic means.

### Short Interfering RNA

US Patent Application 20040023390 (the entire contents of which are hereby incorporated by reference as if fully set forth herein) teaches that double-stranded RNA (dsRNA) also called short interfering RNA (siRNA) herein, can induce sequence-specific post-transcriptional gene silencing in many organisms by a process known as RNA interference (RNAi). Recent work suggests that RNA fragments are the sequence-specific mediators of RNAi (Elbashir et al., 2001). Interference of gene expression by these short interfering RNA (siRNA, usually about 19-21 nucleotides long) is now recognized as a naturally occurring strategy for silencing genes in C. elegans, Drosophila, plants, and in mouse embryonic stem cells, oocytes and early embryos (Cogoni et al., 1994; Baulcombe, 1996; Kennerdell, 1998; Timmons, 1998; Waterhouse et al., 1998; Wianny and Zernicka-Goetz, 2000; Yang et al., 2001; Svoboda et al., 2000, the entire contents of which are hereby incorporated by reference as if fully set forth herein).

In mammalian cell culture, a siRNA-mediated reduction in gene expression has been accomplished by transfecting cells with synthetic RNA nucleic acids (Caplan et al., 2001; Elbashir et al., 2001. The 20040023390 application, the entire contents of which are hereby incorporated by reference as if fully set forth herein, provides methods using a viral vector containing an expression cassette containing a pol II promoter operably-linked to a nucleic acid sequence encoding a short interfering RNA molecule (siRNA) targeted against a gene of interest.

As used herein RNAi is the process of RNA interference. A typical mRNA produces approximately 5,000 copies of a protein. RNAi is a process that interferes with or significantly reduces the number of protein copies made by an mRNA. For example, a double-stranded short interfering RNA (siRNA) molecule is engineered to complement and match the protein-encoding nucleotide sequence of the target mRNA to be interfered with using methods known in the art and described herein. Following intracellular delivery, the siRNA molecule associates with a RNA-induced silencing complex (RISC). The siRNA-associated RISC binds the target mRNA through a base-pairing interaction and degrades it. The RISC remains capable of degrading additional copies of the targeted mRNA. Other forms of RNA can be used such as short hairpin RNA and longer RNA molecules. Longer molecules cause cell death, for example by instigating apoptosis and inducing an interferon response. Cell death was the major hurdle to achieving RNAi in mammals because dsRNAs longer than 30 nucleotides activated defense mechanisms that resulted in non-specific degradation of RNA transcripts and a general shutdown of the host cell. Using from about 20 to about 29 nucleotide siRNAs to mediate gene-specific suppression in mammalian cells has apparently overcome this obstacle. These siRNAs are long enough to cause gene suppression but not of a length that induces an interferon response.

### Percent Identity Determinations

Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from the U.S. government's National Center for Biotechnology Information web site (World Wide Web at ncbi.nlm.nih.gov). Instructions explaining how to use the Bl2seq program can be found in the readme file accompanying BLASTZ. Details of the BLASTZ method are set forth in US Patent Published Application Serial No. 0060088828, Harris; Peter C., et al., entitled, Polycystic kidney disease nucleic acids and proteins, the entire contents of which are hereby incorporated by reference as if fully set forth herein.

### Production of Isolated PIM-1 KINASE Nucleic Acid Molecules

Isolated nucleic acid molecules of the invention can be produced by standard techniques, including, without limitation, common molecular cloning and chemical nucleic acid synthesis techniques. For example, polymerase chain reaction (PCR) techniques can be used to obtain an isolated PIM-1 KINASE nucleic acid molecule. PCR refers to a procedure or technique in which target nucleic acids are enzymatically amplified. Sequence information from the ends of the region of interest or beyond typically is employed to design nucleic acid primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, Ed. by Dieffenbach, C. and Dveksler, G, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication or nucleic acid sequence-based amplification also can be used to obtain isolated nucleic acids. See, for example, Lewis (1992) Genetic Engineering News 12(9):1; Guatelli et al. (1990) Proc. Natl. Acad. Sci. USA 87:1874-1878; and Weiss (1991) Science 254:1292-1293.

In one embodiment, a primer is a single-stranded or double-stranded nucleic acid that typically is 10 to 50 nucleotides in length, and when combined with mammalian genomic DNA and subjected to PCR conditions, is capable of being extended to produce a nucleic acid product corresponding to a region of an PIM-1 KINASE nucleic acid molecule. Typically, a Pim-1 KINASE PCR product is 1235 nucleotides in length (e.g., 30, 35, 50, 100, 250, 500, 1000, 1500, or 1650 nucleotides in length). Primers such as tagcctcctgccccgcggcgc; ctatttgctgggccccggcgacag are particularly useful for producing Pim-1 KINASE PCR products are particularly useful for producing PIM-1 KINASE PCR products. Specific regions of mammalian DNA can be amplified (i.e., replicated such that multiple exact copies are produced) when a pair of nucleic acid primers is used in the same PCR reaction, wherein one primer contains a nucleotide sequence from the coding strand of an PIM-1 KINASE nucleic acid and the other primer contains a nucleotide sequence from the non-coding strand of an PIM-1 KINASE nucleic acid. The “coding strand” of a nucleic acid is the nontranscribed strand, which has the same nucleotide sequence as the specified RNA transcript (with the exception that the RNA transcript contains uracil in place of thymidine residues), while the “non-coding strand” of a nucleic acid is the strand that serves as the template for transcription.

A single PCR reaction mixture may contain one pair of nucleic acid primers. Alternatively, a single reaction mixture may contain a plurality of nucleic acid primer pairs, in which case multiple PCR products can be generated. Each primer pair can amplify, for example, one exon or a portion of one exon. Intron sequences also can be amplified.

Nucleic acid primers can be incorporated into compositions. Typically, a composition of the invention will contain a first nucleic acid primer and a second nucleic acid primer, each 10 to 50 nucleotides in length, which can be combined with genomic DNA from a mammal and subjected to PCR conditions as set out below, to produce a nucleic acid product that corresponds to PIM-1 KINASE nucleic acid molecule or a region thereof. A composition also may contain buffers and other reagents necessary for PCR (e.g., DNA polymerase or nucleotides). Furthermore, a composition may contain one or more additional pairs of nucleic acid primers (e.g., 3, 13, 16, or 23 primer pairs), such that multiple nucleic acid products can be generated.

Specific PCR conditions typically are defined by the concentration of salts (e.g., MgCl2) in the reaction buffer, and by the temperatures utilized for melting, annealing, and extension. Specific concentrations or amounts of primers, templates, deoxynucleotides (dNTPs), and DNA polymerase also may be set out. For example, PCR conditions with a buffer containing 2.5 mM MgCl2, and melting, annealing, and extension temperatures of 94 degrees C., 44-65 degrees C., and 72 degrees C., respectively, are particularly useful. Under such conditions, a PCR sample can include, for example, 60 ng genomic DNA, 8 mM each primer, 200 pM dNTPs, 1 U DNA polymerase (e.g., AmpliTaq Gold), and the appropriate amount of buffer as specified by the manufacturer of the polymerase (e.g., 1.times. AmpliTaq Gold buffer). Denaturation, annealing, and extension each may be carried out for 30 seconds per cycle, with a total of 25 to 35 cycles, for example. An initial denaturation step (e.g., 94 degrees C. for 2 minutes) and a final elongation step (e.g., 72 degrees C. for 10 minutes) also may be useful.

Isolated nucleic acids of the invention also can be chemically synthesized, either as a single nucleic acid molecule (e.g., using automated DNA synthesis in the 3′ to 5′ direction using phosphoramidite technology) or as a series of nucleic acids. For example, one or more pairs of long nucleic acids (e.g., >100 nucleotides) can be synthesized that contain the desired sequence, with each pair containing a short segment of complementarity (e.g., about 15 nucleotides) such that a duplex is formed when the nucleic acid pair is annealed. DNA polymerase is used to extend the nucleic acids, resulting in a single, double-stranded nucleic acid molecule per nucleic acid pair, which then can be ligated into a vector.

Pim-1L kinase for use in the screening assays described and claimed herein can include conservative amino acid substitutions. Conservative amino acid substitutions replace an amino acid with an amino acid of the same class, whereas non-conservative amino acid substitutions replace an amino acid with an amino acid of a different class. Conservative amino acid substitutions typically have little effect on the structure or function of a polypeptide. Examples of conservative substitutions include amino acid substitutions within the following groups: glycine and alanine; valine, isoleucine, and leucine; aspartic acid and glutamic acid; asparagine, glutamine, serine, and threonine; lysine, histidine, and arginine; and phenylalanine and tyrosine.

Pim-1L kinase for use in the screening assays described and claimed herein can include non-conservative substitutions that result in a substantial change in the hydrophobicity or the charge of the polypeptide, as long as the substitutions do not remove the biological activity of Pim-1L kinase. Examples of non-conservative substitutions include a basic amino acid for a non-polar amino acid, or a polar amino acid for an acidic amino acid.

The term “purified” as used herein with reference to a polypeptide refers to a polypeptide that either has no naturally occurring counterpart (e.g., a peptidornimetic), has been chemically synthesized and is thus uncontaminated by other polypeptides, or has been separated or purified from other cellular components by which it is naturally accompanied (e.g., other cellular proteins, polynucleotides, or cellular components). Typically, the polypeptide is considered “purified” when it is at least 70% (e.g., 70%, 80%, 90%, 95%, or 99%), by dry weight, free from the proteins and naturally occurring organic molecules with which it naturally associates.

By way of example and not limitation, Pim-1 kinase can be obtained by extraction from a natural source (e.g., from isolated cells, tissues or bodily fluids), by expression of a recombinant nucleic acid encoding the polypeptide, or by chemical synthesis.

Pim-1 kinase of the invention can be produced by, for example, standard recombinant technology, using expression vectors encoding Pim-1 kinase polypeptides. The resulting Pim-1 kinase then can be purified. Expression systems that can be used for small or large scale production of Pim-1 kinase include, without limitation, microorganisms such as bacteria (e.g., E. coli and B. subtilis) transformed with recombinant bacteriophage DNA, plasmid DNA, or cosmid DNA expression vectors containing the nucleic acid molecules of the invention; yeast (e.g., S. cerevisiae) transformed with recombinant yeast expression vectors containing the nucleic acid molecules of the invention; insect cell systems infected with recombinant virus expression vectors (e.g., baculovirus) containing the nucleic acid molecules of the invention; plant cell systems infected with recombinant virus expression vectors (e.g., tobacco mosaic virus) or transformed with recombinant plasmid expression vectors (e.g., Ti plasmid) containing the nucleic acid molecules of the invention; or mammalian cell systems (e.g., primary cells or immortalized cell lines such as COS cells, Chinese hamster ovary cells, HeLa cells, human embryonic kidney 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids of the invention.

Suitable methods for purifying the polypeptides of the invention can include, for example, affinity chromatography, immunoprecipitation, size exclusion chromatography, and ion exchange chromatography. See, for example, Flohe et al. (1970) Biochlim. Biophys. Acta. 220:469-476, or Tilgmann et al. (1990) FEBS 264:95-99. The extent of purification can be measured by any appropriate method, including but not limited to: column chromatography, polyacrylamide gel electrophoresis, or high-performance liquid chromatography. Pim-1 kinase also can be “engineered” to contain a tag sequence described herein that allows the polypeptide to be purified (e.g., captured onto an affinity matrix). Immunoaffinity chromatography also can be used to purify Pim-1 kinase polypeptides.

### Antibodies

The invention also provides for the therapeutic use of antibodies having specific binding activity for BCRP. Monoclonal anti-BCRP antibody is available commercially: (clone BXP-21, Chemicon). “Antibody” or “antibodies” include intact molecules as well as fragments thereof that are capable of binding to an epitope of a BCRP polypeptide. The term “epitope” refers to an antigenic determinant on an antigen to which an antibody binds. Epitopes usually consist of chemically active surface groupings of molecules such as amino acids or sugar side chains, and typically have specific three-dimensional structural characteristics, as well as specific charge characteristics. Epitopes generally have at least five contiguous amino acids. The terms “antibody” and “antibodies” include polyclonal antibodies, monoclonal antibodies, humanized or chimeric antibodies, single chain Fv antibody fragments, Fab fragments, and F(ab)₂ fragments. Polyclonal antibodies are heterogeneous populations of antibody molecules that are specific for a particular antigen, while monoclonal antibodies are homogeneous populations of antibodies to a particular epitope contained within an antigen. Monoclonal antibodies are particularly useful.

In general, a BCRP polypeptide is produced as described above, i.e., recombinantly, by chemical synthesis, or by purification of the native protein, and then used to immunize animals. Various host animals including, for example, rabbits, chickens, mice, guinea pigs, and rats, can be immunized by injection of the protein of interest to make polyclonal antibodies using well known methods in the art. Depending on the host species, adjuvants can be used to increase the immunological response and include Freund's adjuvant (complete and/or incomplete), mineral gels such as aluminum hydroxide, surface-active substances such as lysolecithin, pluronic polyols, polyanions, peptides, oil emulsions, keyhole limpet hemocyanin, and dinitrophenol. Polyclonal antibodies are contained in the sera of the immunized animals.

A chimeric antibody is a molecule in which different portions are derived from different animal species, such as those having a variable region derived from a mouse monoclonal antibody and a human immunoglobulin constant region. Chimeric antibodies can be produced through standard techniques.

Antibody fragments that have specific binding affinity for BCRP can be generated by known techniques. Such antibody fragments include, but are not limited to, F(ab′)₂ fragments that can be produced by pepsin digestion of an antibody molecule, and Fab fragments that can be generated by deducing the disulfide bridges of F(ab′)₂ fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778.

Once produced, antibodies or fragments thereof can be tested for recognition of a BCRP polypeptide by standard immunoassay methods including, for example, enzyme-linked immunosorbent assay (ELISA) or radioimmuno assay (RIA). See, Short Protocols in Molecular Biology eds. Ausubel et al., Green Publishing Associates and John Wiley & Sons (1992). Suitable antibodies typically have equal binding affinities for recombinant and native proteins.

Fragments of antibodies that bind to and inactivate the 44 kilodalton isoform of Pim-1 kinase and that can enter a cell (intracellular antibody fragments) expressing the 44 kD isoforms, bind to it and inhibit its activation of Etk or binding to an ABC transporter also come within the scope of this invention.

Autoantibodies are naturally occurring antibodies directed to an antigen which the individual's immune system recognizes as foreign even though that antigen originated in the individual. They may be present in the circulation as circulating free antibodies or in the form of circulating immune complexes consisting of the autoantibodies bound to their target antigen.

As used herein with respect to nucleic acids “isolated” means any of a) amplified in vitro by, for example, polymerase chain reaction (PCR), b) recombinantly produced by cloning, c) purified by, for example, gel separation, or d) synthesised, such as by chemical synthesis.

The nucleic acids or nucleic acids according to the invention may carry a revealing label. Suitable labels include radioisotopes such as .sup.32P or .sup.35S, enzyme labels or other protein labels such as biotin or fluorescent markers. Such labels may be added to the nucleic acids or nucleic acids of the invention and may be detected using known techniques per se.

### Drug Formulations

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Nucleic acids with at least one 2′-O-methoxyethyl modification are believed to be particularly useful for oral administration.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, destran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO94/20078, WO/94/23701 and WO96/06638). The amount of active compound contained within a sustained release formulation depends upon the site of implantation, the rate and expected duration of release and the nature of the condition to be treated or prevented.

### EXAMPLES

### Example 1

### Plasmid Constructs and Antibodies

The full-length human 44 kD Pim-1 cDNA was amplified by PCR with a human EST clone (ATCC) as the template. The PCR products were subcloned into the pcDNA3 based vector to replace the Flag-tagged murine p33 Pim-1 (kindly provided by Dr Hirano (24)) by restriction digestion with EcoR I/Xba I. All human Pim-1 constructs contain the N-terminal Flag-tag. To generate the kinase-inactive Pim-1 mutant, the lysine (K or Lys) residue at position 158 of Pim-1L was mutated to methionine (M or Met) via nucleic acid-directed mutagenesis with the forward mutagenic primer 5′-GCCGGTGGCCATCATGCACGTGGAGAAGG-3′, and its reverse primer by using the Quickchange Mutagenesis Kit (Stratagene). The mutants containing deletion (Pim-1LΔP in which the first 15 amino acids are deleted) or mutation (Pim-1LPA in which Proline (P or Pro) 2, 5, 8 and 11 are substituted by Alanines (A or Ala)) of the PXXP motifs of human Pim-1L were generated. All mutations were confirmed by sequencing. T7-tagged Etk and its mutants T7-EtkKQ were described previously (25). The polyclonal antibody against the Pim-1L was generated by immunizing the rabbits with the purified GST fusion protein containing the first 91 amino acids at the N-terminus of Pim-1L following the standard protocol.

Etk deletion mutants were constructed by standard protocols. Ref: Proc. Natl. Acad. Sci. USA 95, 3644-3649 (1998), the entire contents of which are hereby incorporated by reference as if fully set forth herein. Monoclonal anti-BCRP antibody is available commercially (clone BXP-21, Chemicon).

### Example 2

### Cell Culture and Transfection

The tissue arrays were purchased from Zymed (cat# 75-4063). All cell lines (except for CWR-R1) used in this study were purchased from American Tissue Culture Collections. CWR-R1 cells were kindly provided by Dr. C. W. Gregory (26). 293T and COS-1 cells were maintained in DMEM supplemented with 10% fetal bovine serum. LNCaP and PC3 cells were maintained in RPMI1640 supplemented with 10% fetal bovine serum. 22Rv1 and CWR-R1 cells were maintained in RPMI1640 supplemented with 10% heat-inactivated fetal bovine serum. Transfections were performed by using Fugene 6 (Roche), Lipofectamine 2000 (GIBCO/BRL) or the calcium phosphate precipitation method (Biological Mimetics Inc) according to the manufacturer's instructions.

### Example 3

### GST Pull-Down Assay

GST fusion proteins were expressed and purified as previously described^{19,21}. Briefly, the GST fusion proteins were pulled down by glutathione beads at 4° C. for 1 hour and then washed three times with the lysis buffer (27). The immobilized GST fusion proteins were incubated with the lysates of 293T cells transfected with the Flag-tagged Pim-1 for 1 hour at 4° C. The beads were washed with the lysis buffer four times and then the protein complexes were loaded in 10% SDS/PAGE, followed by immunoblotting with anti-Flag antibody.

### Example 4

### Immunoprecipitation and Western Blot

The transfected cells were lysed in the buffer (20 mM TrisHCl pH 7.4, 150 mM NaCl, 1 mM EDTA, 1 mM EGTA, 1% Triton X-100, 1 mM Na₃VO₄, 1 mg/ml aprotinin, 1 mg/ml leupterin and 1 mM PMSF). Insoluble material was removed by centrifugation, and antibodies were added to lysates and incubated for 1-3 hour at 4° C. The immunocomplexes were collected by using protein A or protein G-sepharose beads, and the beads were then washed extensively for three times at 4° C. with the lysis buffer. Immunoblotting was performed as previously described (27). Briefly, blots were incubated with primary antibodies, 1:5000 dilution of anti-T7 tag, 1:2000 dilution of anti-phosphotyrosine, 1:2000 dilution of anti-Flag tag, 1:100 dilution of anti-Pim1 at room temperature for 1 h, and followed by detection with horseradish peroxidase-conjugated secondary antibody.

### Example 5

### In Vitro Kinase Assays

The Etk IVK assays were carried out as described previously (19). Briefly, the Etk immunoprecipitates were washed twice with the kinase buffer (50 mM Tris.HCl, pH 8.0, 10 mM MnCl₂) and then incubated at room temperature with kinase buffer containing 2 μg of GST-Gab1CT and 200 μM ATP. The reaction was terminated by adding the equal volume of 2×SDS sample buffer and boiling for 10 min. The reaction mixtures were separated by 10% SDS-polyacrylamide gel electrophoresis. The tyrosine phosphorylation of Gab1CT was detected with anti-phosphotyrosine antibody (4G10).

### Example 6

### Immunofluorescence and Immunohistochemical Staining and Confocal Microscopy

LNCaP cells were seeded on coverslips and transfected with 0.5 μg DNA/10⁵ cells by the Lipofectamine 2000 transfection reagent (GIBCO/BRL). At 48 hour post-transfection, the cells were fixed in 3.7% paraformaldehyde for 15 min. Immunostaining was performed by incubating the slides with 1:200 dilution of anti-Flag monoclonal antibody (M2) for 45 min and/or with 1:200 dilution of anti-Etk for 1 hour at room temperature, followed by incubation with the Rhodamine-conjugated goat anti-mouse antibody and the FITC-conjugated goat anti-rabbit antibody for 45 min at room temperature. The slides were then washed and mounted with Vectashield (Vector Laboratories). The stained slides were examined by using an inverted microscope under a 60× oil immersion objective and scanned with a laser confocal system. The human prostate tissue arrays containing 20 of paraffin-embedded benign prostate tissue samples and 20 of prostate tumor tissues were purchased from Zymed. The Standard biotin-avidin-complex immunohistochemistry was performed according to the protocol provided by the manufacturer (Vector Laboratories).

### Example 7

### Drug Sensitivity Assay

LNCaP cells were infected with the lentiviruses encoding the proteins as indicated by using the protocol described previously (19). At 48 hour post-infection, the cells were seeded into the 96 well plates (3×10⁴/well). Doxorubicin or mitoxantrone was added in the medium after 24 h. The effects of doxorubicin on the viability of cells were measured after 20-48 hour by the WST-1 assay (Roche Molecular Biochemicals). The viability rates are expressed as means±SD of the triplicate for each experiment. An aliquot of cells was lysed and followed by immunoblotting with anti-T7 or anti-Flag to monitor the transfection efficiency.

### Example 8

### Establishment of Prostate Cancer Cell Lines with Acquired Resistance to Docetaxel or Mitoxantrone

LNCaP is an AR-positive prostate cancer cell line which is sensitive to various chemotherapeutic drugs. Therefore, the LNCaP cell line was chosen to establish a cell line with acquired resistance to docetaxel (DTX) or mitoxantrone (MX). During a period of 1 month, LNCaP cells were continuously exposed to a variety of concentrations of DTX or MX and cell viability was examined. Concentrations of 1 nM DTX or 10 μM MX were chosen for testing, and viable cells surviving at this proapoptotic concentration were continuously exposed for 3 months in order to establish the cell line.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

### REFERENCES

1. Cuypers, H. T., Selten, G., Quint, W., Zijlstra, M., Maandag, E. R., Boelens, W., van Wezenbeek, P., Melief, C., and Berns, A. Murine leukemia virus-induced T-cell lymphomagenesis: integration of proviruses in a distinct chromosomal region. Cell, 37: 141-150, 1984.
2. von Lindern, M., van Agthoven, T., Hagemeijer, A., Adriaansen, H., and Grosveld, G. The human pim-1 gene is not directly activated by the translocation (6; 9) in acute nonlymphocytic leukemia. Oncogene, 4: 75-79, 1989.
3. Amson, R., Sigaux, F., Przedborski, S., Flandrin, G., Givol, D., and Telerman, A. The human protooncogene product p33pim is expressed during fetal hematopoiesis and in diverse leukemias. Proc Natl Acad Sci USA, 86: 8857-8861, 1989.
4. Breuer, M., Slebos, R., Verbeek, S., van Lohuizen, M., Wientjens, E., and Berns, A. Very high frequency of lymphoma induction by a chemical carcinogen in pim-1 transgenic mice. Nature, 340: 61-63, 1989.
5. van Lohuizen, M., Verbeek, S., Krimpenfort, P., Domen, J., Saris, C., Radaszkiewicz, T., and Berns, A. Predisposition to lymphomagenesis in pim-1 transgenic mice: cooperation with c-myc and N-myc in murine leukemia virus-induced tumors. Cell, 56: 673-682, 1989.
6. Leverson, J. D., Koskinen, P. J., Orrico, F. C., Rainio, E. M., Jalkanen, K. J., Dash, A. B., Eisenman, R. N., and Ness, S. A. Pim-1 kinase and p100 cooperate to enhance c-Myb activity. Mol Cell, 2: 417-425, 1998.
7. Mochizuki, T., Kitanaka, C., Noguchi, K., Muramatsu, T., Asai, A., and Kuchino, Y. Physical and functional interactions between Pim-1 kinase and Cdc25A phosphatase. Implications for the Pim-1-mediated activation of the c-Myc signaling pathway. J Biol Chem, 274: 18659-18666, 1999.
8. Koike, N., Maita, H., Taira, T., Ariga, H., and Iguchi-Ariga, S. M. Identification of heterochromatin protein 1 (HP1) as a phosphorylation target by Pim-1 kinase and the effect of phosphorylation on the transcriptional repression function of HP1 (1). FEBS Lett, 467: 17-21, 2000.
9. Zhao, T., Heyduk, T., and Eissenberg, J. C. Phosphorylation site mutations in heterochromatin protein 1 (HP 1) reduce or eliminate silencing activity. J Biol Chem, 276: 9512-9518, 2001.
10. Aho, T. L., Sandholm, J., Peltola, K. J., Mankonen, H. P., Lilly, M., and Koskinen, P. J. Pim-1 kinase promotes inactivation of the pro-apoptotic Bad protein by phosphorylating it on the Ser112 gatekeeper site. FEBS Lett, 571: 4349, 2004.
11. Yan, B., Zemskova, M., Holder, S., Chin, V., Kraft, A., Koskinen, P. J., and Lilly, M. The PIM-2 kinase phosphorylates BAD on serine 112 and reverses BAD-induced cell death.
Biol Chem, 278: 45358-45367, 2003.
12. Dhanasekaran, S. M., Barrette, T. R., Ghosh, D., Shah, R., Varambally, S., Kurachi, K., Pienta, K. J., Rubin, M. A., and Chinnaiyan, A. M. Delineation of prognostic biomarkers in prostate cancer. Nature, 412: 822-826, 2001.
13. Wang, S., Gao, J., Lei, Q., Rozengurt, N., Pritchard, C., Jiao, J., Thomas, G. V., Li, G., Roy-Burman, P., Nelson, P. S., Liu, X., and Wu, H. Prostate-specific deletion of the murine Pten tumor suppressor gene leads to metastatic prostate cancer. Cancer Cell, 4: 209-221, 2003.
14. Ellwood-Yen, K., Graeber, T. G., Wongvipat, J., Iruela-Arispe, M. L., Zhang, J., Matusik, R., Thomas, G. V., and Sawyers, C. L. Myc-driven murine prostate cancer shares molecular features with human prostate tumors. Cancer Cell, 4: 223-238, 2003.
15. Valdman, A., Fang, X., Pang, S. T., Ekman, P., and Egevad, L. Pim-1 expression in prostatic intraepithelial neoplasia and human prostate cancer. Prostate, 60: 367-371, 2004.
16. Volk, E. L., Rohde, K., Rhee, M., McGuire, J. J., Doyle, L. A., Ross, D. D., and Schneider, E. Methotrexate cross-resistance in a mitoxantrone-selected multidrug-resistant MCF7 breast cancer cell line is attributable to enhanced energy-dependent drug efflux. Cancer Res, 60: 3514-3521, 2000.
17. Saris, C. J., Domen, J., and Berns, A. The pim-1 oncogene encodes two related protein-serine/threonine kinases by alternative initiation at AUG and CUG. Embo J, 10: 655-664, 1991.
18. Ishibashi, Y., Maita, H., Yano, M., Koike, N., Tamai, K., Ariga, H., and Iguchi-Ariga, S. M. Pim-1 translocates sorting nexin 6/TRAF4-associated factor 2 from cytoplasm to nucleus. FEBS Lett, 506: 33-38, 2001.
19. Kim, O., Jiang, T., Xie, Y., Guo, Z., Chen, H., and Qiu, Y. Synergism of cytoplasmic kinases in IL6-induced ligand-independent activation of androgen receptor in prostate cancer cells. Oncogene, 23: 1838-1844, 2004.
20. Jiang, T., Guo, Z., Dai, B., Kang, M., Ann, D. K., Kung, H. J., and Qiu, Y. Bi-directional regulation between tyrosine kinase Etk/BMX and tumor suppressor p53 in response to DNA damage. J Biol Chem, 279: 50181-50189, 2004.
21. Wang, Z., Bhattacharya, N., Mixter, P. F., Wei, W., Sedivy, J., and Magnuson, N. S. Phosphorylation of the cell cycle inhibitor p21 Cip1/WAF1 by Pim-1 kinase. Biochim Biophys Acta, 1593: 45-55, 2002.
22. Chen, R., Kim, O., Li, M., Xiong, X., Guan, J. L., Kung, H. J., Chen, H., Shimizu, Y., and Qiu, Y. Regulation of the PH-domain-containing tyrosine kinase Etk by focal adhesion kinase through the FERM domain. Nat Cell Biol, 3: 439-444, 2001.
23. Doyle, L. A. and Ross, D. D. Multidrug resistance mediated by the breast cancer resistance protein BCRP (ABCG2). Oncogene, 22: 7340-7358, 2003.
24. Friedmann, M., Nissen, M. S., Hoover, D. S., Reeves, R., and Magnuson, N. S. Characterization of the proto-oncogene pim-1: kinase activity and substrate recognition sequence. Arch Biochem Biophys, 298: 594-601, 1992.
25. Shirogane, T., Fukada, T., Muller, J. M., Shima, D. T., Hibi, M., and Hirano, T. Synergistic roles for Pim-1 and c-Myc in STAT3-mediated cell cycle progression and antiapoptosis. Immunity, 11: 709-719, 1999.
26. Qiu, Y., Robinson, D., Pretlow, T. G., and Kung, H. J. Etk/Bmx, a tyrosine kinase with a pleckstrin-homology domain, is an effector of phosphatidylinositol 3′-kinase and is involved in interleukin 6-induced neuroendocrine differentiation of prostate cancer cells. Proc Natl Acad Sci USA, 95: 3644-3649, 1998.
27. Gregory, C. W., Johnson, R. T., Jr., Mohler, J. L., French, F. S., and Wilson, E. M. Androgen receptor stabilization in recurrent prostate cancer is associated with hypersensitivity to low androgen. Cancer Res, 61: 2892-2898, 2001.
28. Kim, O., Yang, J., and Qiu, Y. Selective Activation of Small GTPase RhoA by Tyrosine Kinase Etk through Its Pleckstrin Homology Domain. J Biol Chem, 277: 30066-30071, 2002.
29. Xie, Y., Xu, K., Dai, B., Guo, Z., Jiang, T., Chen, H., and Qiu, Y. The 44 kDa Pim-1 kinase directly interacts with tyrosine kinase Etk//BMX and protects human prostate cancer cells from apoptosis induced by chemotherapeutic drugs. Oncogene, 25: 70-78, 2006.

What is claimed is:

1-58. (canceled)

59. An isolated nucleic acid that inhibits expression of the 44 kilodalton isoform of PIM-1 kinase, selected from the group comprising short interfering RNAs and antisense nucleic acids.

60. The isolated nucleic acids as recited in claim 59, wherein the antisense nucleic acids are sufficiently complementary to DNA encoding the 44 kilodalton isoform of PIM-1 kinase identified in SEQ ID NO. 1 or to messenger RNA encoding the 44 kilodalton isoform of PIM-1kinase identified in SEQ ID NO. 2, to permit specific hybridization under physiologic conditions.

61. The isolated nucleic acids as recited in claim 59, wherein the short interfering RNA comprises the sequence set forth in SEQ ID NO. 3 or a variant thereof.

62. The isolated nucleic acids according to claim 59, wherein the antisense nucleic acids are from about 8 to about 50 nucleobases in length.

63. A method of treating cancer in an animal, comprising administering to the animal a therapeutic or prophylactic amount of an isolated nucleic acid that inhibits expression of the 44 kilodalton isoform of PIM-1 kinase, selected from the group comprising short interfering RNAs and antisense nucleic acids.

64. The method as recited in claim 63, wherein the antisense nucleic acids are sufficiently complementary to DNA encoding the 44 kilodalton isoform of PIM-1 kinase identified in SEQ ID NO. 1 or to messenger RNA encoding the 44 kilodalton isoform of PIM-1kinase identified in SEQ ID NO. 2, to permit specific hybridization under physiologic conditions.

65. The method as recited in claim 63, wherein the short interfering RNA comprises the sequence set forth in SEQ ID NO. 3 or a variant thereof.

66. The method as recited in claim 63, wherein the antisense nucleic acids are from about 8 to about 50 nucleobases in length.

67. The method as in claim 63, wherein the cancer is hematopoietic or prostate cancer.

68. A method of inhibiting the expression of the 44 kilodalton isoform of Pim-1 kinase in cells or tissues from an organism, comprising contacting the cells or tissues in vitro or in vivo with an isolated nucleic acid that inhibits expression of the 44 kilodalton isoform of PIM-1 kinase, selected from the group comprising short interfering RNAs and antisense nucleic acids.

69. The method as recited in claim 68, wherein the antisense nucleic acids are sufficiently complementary to DNA encoding the 44 kilodalton isoform of PIM-1 kinase identified in SEQ ID NO. 1 or to messenger RNA encoding the 44 kilodalton isoform of PIM-1kinase identified in SEQ ID NO. 2, to permit specific hybridization under physiologic conditions.

70. The method as recited in claim 68, wherein the short interfering RNA comprises the sequence set forth in SEQ ID NO. 3 or a variant thereof.

71. The method as recited in claim 68, where the antisense nucleic acids are from about 8 to about 50 nucleobases in length.

72. The method according to claim 59, wherein the antisense nucleic acid molecule comprises RNA or DNA.
